SEQUENCE LISTING (58 sequences)

SEQ ID NO: 1 - DNA, 25 bp, synthetic construct
  source 1..25: mol_type = other DNA
attcaaccaatcataaagatattgg

SEQ ID NO: 2 - DNA, 26 bp, synthetic construct
  source 1..26: mol_type = other DNA
ttctcaaccaaccacaaagacattgg

SEQ ID NO: 3 - DNA, 26 bp, synthetic construct
  source 1..26: mol_type = other DNA
ttctcaaccaaccacaargayatygg

SEQ ID NO: 4 - DNA, 25 bp, synthetic construct
  source 1..25: mol_type = other DNA
ggtcaacaaatcataaagatattgg

SEQ ID NO: 5 - DNA, 25 bp, synthetic construct
  source 1..25: mol_type = other DNA
ggtcaacaaatcataaagayatygg

SEQ ID NO: 6 - DNA, 22 bp, synthetic construct
  source 1..22: mol_type = other DNA
tnttmtcaacnaaccacaaaga

SEQ ID NO: 7 - DNA, 26 bp, synthetic construct
  source 1..26: mol_type = other DNA
ttctccaaccacaaagacattggcac

SEQ ID NO: 8 - DNA, 26 bp, synthetic construct
  source 1..26: mol_type = other DNA
tcyactaatcataaagatattggyac

SEQ ID NO: 9 - DNA, 20 bp, synthetic construct
  source 1..20: mol_type = other DNA
agggcaakyctggtgccagc

SEQ ID NO: 10 - DNA, 26 bp, synthetic construct
  source 1..26: mol_type = other DNA
taaacttctggatgtccaaaaaatca

SEQ ID NO: 11 - DNA, 26 bp, synthetic construct
  source 1..26: mol_type = other DNA
tagacttctgggtggccraaraayca

SEQ ID NO: 12 - DNA, 26 bp, synthetic construct
  source 1..26: mol_type = other DNA
tagacttctgggtggccaaagaatca

SEQ ID NO: 13 - DNA, 26 bp, synthetic construct
  source 1..26: mol_type = other DNA
taaacttcagggtgaccaaaaaatca

SEQ ID NO: 14 - DNA, 26 bp, synthetic construct
  source 1..26: mol_type = other DNA
taaacttcagggtgaccaaaraayca

SEQ ID NO: 15 - DNA, 23 bp, synthetic construct
  source 1..23: mol_type = other DNA
acttctggrtgkccaaaraatca

SEQ ID NO: 16 - DNA, 20 bp, synthetic construct
  source 1..20: mol_type = other DNA
tcdggrtgnccraaraayca

SEQ ID NO: 17 - DNA, 19 bp, synthetic construct
  source 1..19: mol_type = other DNA
grcggtatctratcgyctt

SEQ ID NO: 18 - DNA, 28 bp, synthetic construct
  source 1..28: mol_type = other DNA
cattttchactaaycataargatattgg

SEQ ID NO: 19 - DNA, 25 bp, synthetic construct
  source 1..25: mol_type = other DNA
tataaacytcdggatgnccaaaaaa

SEQ ID NO: 20 - DNA, 19 bp, synthetic construct
  source 1..19: mol_type = other DNA
gtaaatatatgrtgdgctc

SEQ ID NO: 21 - DNA, 26 bp, synthetic construct
  source 1..26: mol_type = other DNA
cccggtaaaattaaaatataaacttc

SEQ ID NO: 22 - DNA, 26 bp, synthetic construct
  source 1..26: mol_type = other DNA
atgtcaccamaaacagaractaaagc

SEQ ID NO: 23 - DNA, 20 bp, synthetic construct
  source 1..20: mol_type = other DNA
gtraaatcaagbccaccrcg

SEQ ID NO: 24 - DNA, 21 bp, synthetic construct
  source 1..21: mol_type = other DNA
acaytcataammhgcyctacc

SEQ ID NO: 25 - DNA, 22 bp, synthetic construct
  source 1..22: mol_type = other DNA
ccdtygagtctctgcacctatc

SEQ ID NO: 26 - DNA, 20 bp, synthetic construct
  source 1..20: mol_type = other DNA
cgraatyggtagacgctacg

SEQ ID NO: 27 - DNA, 22 bp, synthetic construct
  source 1..22: mol_type = other DNA
aacttaaaggaattgacggaag

SEQ ID NO: 28 - DNA, 20 bp, synthetic construct
  source 1..20: mol_type = other DNA
ccttcygcaggttcacctac

SEQ ID NO: 29 - DNA, 18 bp, synthetic construct
  source 1..18: mol_type = other DNA
ascygyggtaaywccagc

SEQ ID NO: 30 - DNA, 18 bp, synthetic construct
  source 1..18: mol_type = other DNA
tchnhgnatttcaccnct

SEQ ID NO: 31 - DNA, 19 bp, synthetic construct
  source 1..19: mol_type = other DNA
ggtcatttagaggaagtaa

SEQ ID NO: 32 - DNA, 17 bp, synthetic construct
  source 1..17: mol_type = other DNA
tccgtaggtgaacctgc

SEQ ID NO: 33 - DNA, 22 bp, synthetic construct
  source 1..22: mol_type = other DNA
ggaagtaaaagtcgtaacaagg

SEQ ID NO: 34 - DNA, 20 bp, synthetic construct
  source 1..20: mol_type = other DNA
tcctscgcttattgatatgc

SEQ ID NO: 35 - DNA, 21 bp, synthetic construct
  source 1..21: mol_type = other DNA
cgcctscscttantdatatgc

SEQ ID NO: 36 - DNA, 22 bp, synthetic construct
  source 1..22: mol_type = other DNA
caagrgacttrtacacggtcca

SEQ ID NO: 37 - DNA, 23 bp, synthetic construct
  source 1..23: mol_type = other DNA
gtcgtaacaaggtttccgtaggt

SEQ ID NO: 38 - DNA, 20 bp, synthetic construct
  source 1..20: mol_type = other DNA
cyygyctgayctgrggtcdc

SEQ ID NO: 39 - DNA, 20 bp, synthetic construct
  source 1..20: mol_type = other DNA
tcctccgcttattkatatgc

SEQ ID NO: 40 - DNA, 22 bp, synthetic construct
  source 1..22: mol_type = other DNA
ggaagkaraagtcgtaacaagg

SEQ ID NO: 41 - DNA, 17 bp, synthetic construct
  source 1..17: mol_type = other DNA
ccggttgatccygscrg

SEQ ID NO: 42 - DNA, 17 bp, synthetic construct
  source 1..17: mol_type = other DNA
gttygatymtggctcag

SEQ ID NO: 43 - DNA, 20 bp, synthetic construct
  source 1..20: mol_type = other DNA
tnanacatgcaagtcgrrcg

SEQ ID NO: 44 - DNA, 20 bp, synthetic construct
  source 1..20: mol_type = other DNA
aaactyaaarraattgacgg

SEQ ID NO: 45 - DNA, 18 bp, synthetic construct
  source 1..18: mol_type = other DNA
hgcyttcgccachggtrg

SEQ ID NO: 46 - DNA, 18 bp, synthetic construct
  source 1..18: mol_type = other DNA
tacnvgggtatctaatcc

SEQ ID NO: 47 - DNA, 17 bp, synthetic construct
  source 1..17: mol_type = other DNA
gacgggcggtgwgtrca

SEQ ID NO: 48 - DNA, 20 bp, synthetic construct
  source 1..20: mol_type = other DNA
aaggaggtgatccanccyca

SEQ ID NO: 49 - DNA, 17 bp, synthetic construct
  source 1..17: mol_type = other DNA
gccagcaaccgcggtaa

SEQ ID NO: 50 - DNA, 21 bp, synthetic construct
  source 1..21: mol_type = other DNA
ttggagggcaagtctggtgcc

SEQ ID NO: 51 - DNA, 20 bp, synthetic construct
  source 1..20: mol_type = other DNA
cagccgcggtaattccagct

SEQ ID NO: 52 - DNA, 18 bp, synthetic construct
  source 1..18: mol_type = other DNA
tctggacctggtgagttt

SEQ ID NO: 53 - DNA, 22 bp, synthetic construct
  source 1..22: mol_type = other DNA
gaacccaaacactttggtttcc

SEQ ID NO: 54 - DNA, 20 bp, synthetic construct
  source 1..20: mol_type = other DNA
gtttcccgtaaggcgccgaa

SEQ ID NO: 55 - DNA, 19 bp, synthetic construct
  source 1..19: mol_type = other DNA
gtgartcatcrartytttg

SEQ ID NO: 56 - DNA, 19 bp, synthetic construct
  source 1..19: mol_type = other DNA
ttyrckrcgttcttcatcg

SEQ ID NO: 57 - DNA, 13 bp, synthetic construct
  source 1..13: mol_type = other DNA
agcaatcgcgcac

SEQ ID NO: 58 - DNA, 13 bp, synthetic construct
  source 1..13: mol_type = other DNA
aaccagcgcctag

(19) United States
(12) Patent Application Publication
(10) Pub. No.: US 2026/0242879 A1
(43) Pub. Date: Aug. 20, 2026

(54) KITS AND METHODS FOR BIODIVERSITY RECOVERY IN AN ENVIRONMENTAL SAMPLE

(71) Applicant: BIODIVERSE INC., Brighton, MI (US)

(72) Inventor: Stephen Douglas Russell, Brighton, MI (US)

(21) Appl. No.: 19/660,611

(22) Filed: Apr. 28, 2026

Related U.S. Application Data
(63) Continuation of application No. PCT/US25/52134, filed on Oct. 22, 2025.
(60) Provisional application No. 63/710,223, filed on Oct. 22, 2024.

(51) Int. Cl. C12Q 1/6895; B01L 3/00

(52) U.S. Cl. CPC C12Q 1/6895; B01L 3/5085; B01L 2300/0829; C12Q 2600/16

(57) ABSTRACT

A kit may include a reaction receptacle, for example a well of a multi-well plate, for receiving an environmental sample. A kit may include a plurality of primer sets complementary to a taxonomic barcode region of a genome. Each primer set may be disposed in a reaction receptacle. Each primer set may include a first sequence including a sample tag sequence used to identify each environmental sample. Each primer set may include a plurality of primers including one or more forward primers and one or more reverse primers. The plurality of primers does not form or include obligate 1:1 pairs of forward and reverse primers. The plurality of primers may include a taxonomically informative sequence that is complementary to a region of DNA of a taxonomic group. The taxonomically informative sequence may be used to identify whether the taxonomic group is present in one or more environmental samples.

### CROSS-REFERENCE TO RELATED APPLICATIONS

This application is a continuation of International Patent Application Ser. No. PCT/US2025/052134, filed Oct. 22, 2025, which claims the priority benefit of U.S. Provisional Patent Application Ser. No. 63/710,223, filed Oct. 22, 2024, the contents of each of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### REFERENCE TO SEQUENCE LISTING SUBMITTED ELECTRONICALLY

The present application is being filed along with a Sequence Listing in electronic format. The Sequence Listing is provided as a file entitled 0173-700.600 Biodiverse Labs.xml, created Oct. 10, 2025, which is 51,000 bytes in size. The information in the electronic format of the Sequence Listing is incorporated by reference in its entirety.

### TECHNICAL FIELD

This disclosure relates generally to the field of environmental sampling and analysis, and, more specifically, to the field of biodiversity in environmental samples. Described herein are kits and methods for biodiversity recovery in an environmental sample.

### BACKGROUND

Currently, the world is thought to be undergoing a polycrisis—a series of individual, yet often interrelated series of changes that will have long-term negative effects for global ecosystems. One of the core elements of the current polycrisis is a biodiversity crisis. The health, diversity, and productivity of the nation's natural environments rely on biodiversity. Diverse ecosystems are capable of resisting and adapting to disturbances like wildfires, pests, and climate change. By some estimates, up to 40% of global species may become extinct by the end of this century. Governments, corporations, and individuals from around the world are beginning to take notice of this emerging biodiversity collapse. Global frameworks are beginning to require an assessment of the impact of projects on nature as a core element of future planning. One element of assessing a project's impact on the natural world is to examine the impact on biodiversity.

### SUMMARY

Described herein are kits and methods for generating amplicons based on computationally refined non-obligate 1:1 primer pair pools, designed to increase biodiversity capture. The amplicons are sequenced and processed with permutational demultiplexing.

In some aspects, the techniques described herein relate to a kit for determining a biodiversity of an environmental sample, the kit including: a multi-well plate, wherein each well of the multi-well plate is configured to receive the environmental sample from a plurality of environmental samples; and a plurality of primer sets complementary to a taxonomic region of a genome, each primer set being disposed in a well of the multi-well plate, wherein: each primer includes a first sequence configured to be demultiplexed to identify each environmental sample of the plurality of environmental samples of the multi-well plate, and each primer set includes a plurality of primers including one or more forward primers and one or more reverse primers, wherein: the plurality of primers does not include obligate 1:1 pairs of forward and reverse primers, the plurality of primers includes a second sequence that is complementary to a region of a taxonomic group, and is configured to be demultiplexed to identify one or more taxonomic groups of interest in one or more environmental samples of the plurality of environmental samples.

In some aspects, the techniques described herein relate to a kit for determining a biodiversity of an environmental sample, the kit including: a multi-well plate, wherein each well of the multi-well plate is configured to receive the environmental sample from a plurality of environmental samples; and a plurality of primer sets, each primer set being disposed in a well of the multi-well plate, wherein: each primer set includes a first sequence including a sample tag sequence configured to be demultiplexed to identify each environmental sample of the plurality of environmental samples of the multi-well plate, and each primer set includes a plurality of primers including one or more forward primers and one or more reverse primers, wherein: the plurality of primers does not include obligate 1:1 pairs of forward and reverse primers, a first subset of the plurality of primers includes a first taxonomically informative sequence that is complementary to a first region of a first taxonomic group, a second subset of the plurality of primers includes a second taxonomically informative sequence that is complementary to a second region of a second taxonomic group, and the first taxonomically informative sequence and the second taxonomically informative sequence are configured to be demultiplexed to identify whether one or both of the first taxonomic group and the second taxonomic group are present in one or more environmental samples of the plurality of environmental samples.

In some aspects, the techniques described herein relate to a computer-implemented method, configured to be performed by one or more hardware processors, for determining a biodiversity of an environmental sample, the computer-implemented method including: receiving sequenced DNA data based on amplified DNA from an executed PCR, the amplified DNA having been amplified with a plurality of primer sets, wherein: each primer set includes a plurality of primers including one or more forward primers and one or more reverse primers, the plurality of primers does not include obligate 1:1 pairs of forward primers and reverse primers, and each primer set is specific to one or more taxonomic regions of target DNA; and demultiplexing the sequenced DNA data to identify at least one taxonomic group, wherein the demultiplexing is based on: a sample tag sequence integrated into each primer set, the sample tag sequence being configured to identify each environmental sample, and each permutation of each primer combination that is present in the plurality of primers specific to an individual barcode region.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates an embodiment of a kit and associated method for biodiversity recovery in an environmental sample.

FIG. 2 illustrates a system for biodiversity recovery in an environmental sample.

FIG. 3A illustrates a polymerase chain reaction (PCR) amplification reaction using tagged primers for amplifying DNA in an environmental sample.

FIG. 3B illustrates a first process of a dual-PCR reaction for amplifying DNA in an environmental sample.

FIG. 3C illustrates a second process of a dual-PCR reaction for amplifying DNA in an environmental sample and adding sequencing tags to the amplicons.

FIG. 4 illustrates an embodiment of a computer-implemented method for biodiversity recovery in an environmental sample.

FIG. 5 illustrates an embodiment of a primer pool.

FIG. 6 illustrates a flow diagram of a computer-implemented method for demultiplexing sequencing data.

FIG. 7 is a graphical representation of sequencing data showing number of reads per sequence length for primer pools spanning the ITS, COI, and 18S regions.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Conventionally, environmental DNA (eDNA) metabarcoding research utilizes individual primer pairs in a single PCR reaction to document the life that exists within a given sample. As an example, if a researcher were interested in fungi and insects, one primer pair may be used for fungi and one primer pair may be used for insects. In such examples, four total primers across two separate reactions are used. It is uncommon for research to go beyond six different reaction-primer pair combinations (i.e., 12 total primers) that would analyze six different target loci in six different PCR reactions. This is a historical holdover from Sanger sequencing, where individual sequencing primers were typically used to perform the final sequencing reaction. The kits and methods described herein solve the above technical problem, of using specific primer pair combinations, with a technical solution, of using a plurality of primers that do not form obligate 1:1 pairs in a reaction. As used herein, an “obligate 1:1 pair” can mean that a forward primer and a reverse primer, in a pair, amplify their respective sequences and the sequence between the forward primer and the reverse primer. Further, in a conventional obligate 1:1 pair, the forward primer is generated, structured, or otherwise designed to only work with a single reverse primer, but not work with other primers within a single PCR reaction. In contrast, the kits and methods described herein do not use primers in obligate 1:1 pairs, such that one forward primer can amplify its sequence and the sequence between itself and any number of reverse primers around a target locus and/or across a multiplex reaction targeting multiple loci in a single reaction. Similarly, in the kits and methods described herein, one reverse primer can amplify its sequence and the sequence between itself and any number of forward primers. Accordingly, the plurality of primers, and their various possible combinations, can amplify a plurality of individual target loci in a single reaction. Said another way, the kids and methods described herein use layered multiplex reactions. A standard multiplex reaction targets multiple different loci with 1:1 primer pairs. The layered multiplex reaction described herein incorporates computationally refined pools of multiple forward and reverse primers at each barcode locus. This reduces taxonomic bias, thus improving overall biodiversity capture in the reaction and enables more robust biodiversity assessments with fewer PCR reactions.

The kits and methods described herein provide further technical solutions including capturing more biodiversity with fewer PCR reactions, labor hours, reagents, and consumables, thus significantly lowering the cost of documenting biodiversity.

Described herein are kits that include pools of PCR primers that are mixed in predefined ratios in order to accomplish specific objectives. For example, loci may be biased. There are more bacterial DNA than any other organismal group in an environmental DNA sample, as an example. A primer concentration may be lowered to limit the amount of amplification of one species to at least partially unbias the amplification process. Further, for example, within an environmental DNA sample, fungal DNA may be amplified preferentially to plant DNA. Thus, the primer pools or plurality of primers may be in a 20:1 ratio, a 15:1 ratio, a 10:1 ratio, or a 5:1 ratio, of the primers targeting plants versus targeting fungal loci.

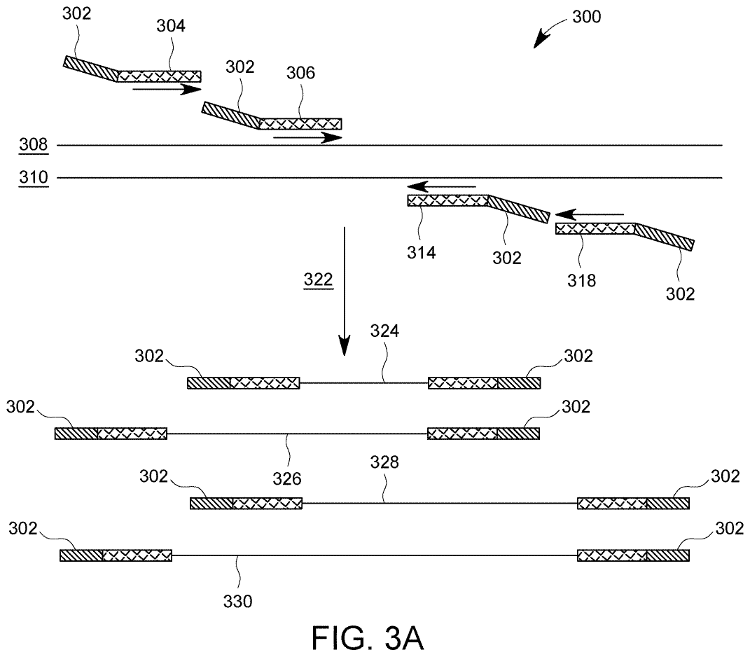

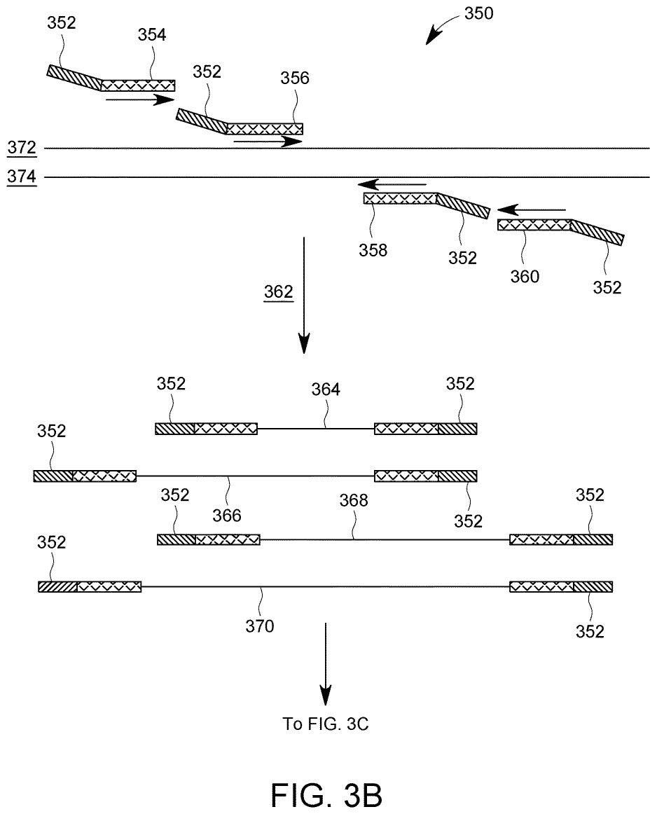

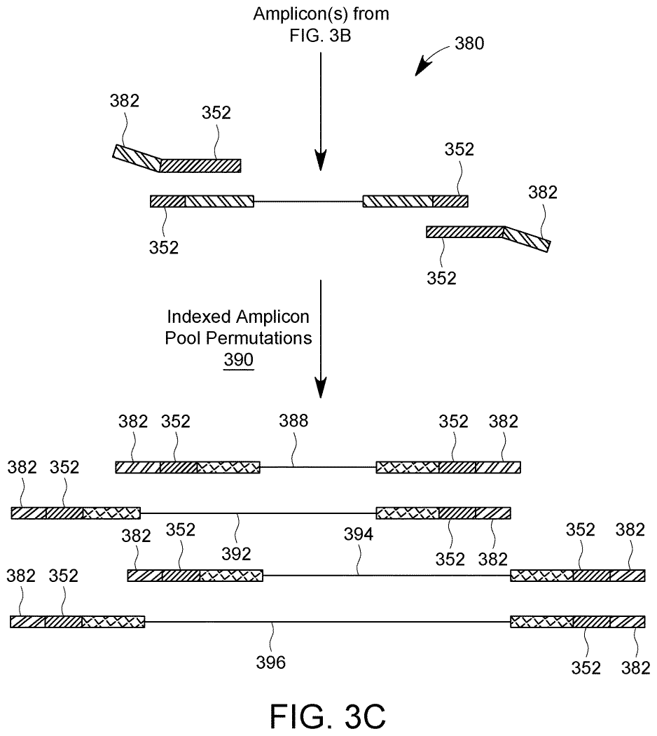

For example, the pools of PCR primers may be in 96 well plate formats (although other reaction receptacle formats are contemplated herein). Each reaction receptacle has a tagged primer set or tagged plurality of primers, for example in single PCR reaction examples (e.g., FIG. 3A), which represents an individual sample in a reaction receptacle or well. In some embodiments, each reaction receptacle has a primer set or a plurality of primers, that may be tagged in a dual-PCR reaction (e.g., FIGS. 3B-3C) so that each primer set or plurality of primers represents an individual sample, in a reaction receptacle or well, once tagged. Further, each primer, primer set, or plurality of primers may be complementary to a sequence or sequences that represents an individual taxonomic group, or one or more taxonomic groups, that may be in the specimen or sample. Alternatively, a subset of each primer set or a subset of the plurality of primers may be complementary to a sequence that represents an individual taxonomic group, or one or more taxonomic groups, that may be in the specimen or sample.

As used herein, a “tag,” an “index,” or a “barcode” may refer to a short sequence of nucleotides that is added to DNA fragments or a DNA sequence (e.g., primers) to distinguish or label the DNA fragment during sequencing or analysis. As described herein, an index or tag may be used in PCR and/or DNA sequencing, such as next generation sequencing (NGS). In some embodiments, after sequencing, the tag may be used to assign reads back to their corresponding samples, for example an environmental sample. The tag may be attached to one or both ends of the DNA fragment (e.g., primer) during PCR amplification using indexed primers (i.e., primers having the tag sequence). In some embodiments, as shown in FIG. 3A, a first tag or sample tag may be used to identify an environmental sample, for example when a plurality of environmental samples is analyzed. In some embodiments, the sample tag (e.g., the tag that identifies the environmental sample) does not anneal to the target DNA but is instead added to the amplicon (i.e., amplified sequence) to aid in the detection, differentiation, or labeling of the amplified products. The sample tag sequence may be an about 8 bp to about 15 bp, an about 10 base pair to about 20 base pair, an about 8 base pair to an about 20 base pair, an about 8 base pair to an about 24 base pair, an about 10 base pair to an about 15 base pair, or an about 15 base pair to an about 20 base pair, etc. unique base pair sequence. Exemplary, non-limiting tag sequences are shown as SEQ ID NOs. 57-58. In some embodiments, a primer of the plurality of primers includes a taxonomic sequence (e.g., specific for a taxonomic sequence) that may anneal to the target DNA such that the complementary taxonomic sequence is present in the environmental DNA and represents one or more taxonomies.

As used herein, “sample” or “environmental sample” may include a specimen-based sample, a physical sample of a lifeform, a voucher specimen (i.e., specimen preserved and/or stored), a soil sample, an air sample, a water sample, a gut sample from an organism, a physical specimen of a lifeform, a sample isolated from a tool having interacted with an element in an environment, a sample from a built environment, a microbiome sample (internal or external microbiome), a sediment sample, an ice sample, a permafrost sample, a snow sample, a biofilm sample, a wood sample, a surface swab of a natural element, and the like.

In some embodiments, one or more primers or a plurality of primers in a reaction may be refined for position within a target locus, a specificity for a target organismal group(s), a specificity at a 3′ or 3 prime end, a melting point, a guanine-cytosine (GC) content, a dimerization potential, a hairpin formation potential, a target final primer concentration, high specificity to a target sequence, and the like. The position may be refined for final amplicon length and to ensure broad coverage across the target locus. For example, conventionally, some primers may be used to create subsets or shorter amplicons. Adding a conventional primer, such as this, to the plurality of primers described herein would limit an amplicon size of the entire primer pool or plurality of primers. A primer, one or more primers, a plurality of primers, at least one of the primers, etc. may include a degenerate base, one or more degenerate bases, a plurality of degenerate bases, at least one degenerate base, etc. to accommodate sequence variability in the target sequence. According to the International Union of Pure and Applied Chemistry (IUPAC) nucleotide code, specific letters represent one or more nucleotides:
N: Represents A, T, C, or G (any base)
R: Represents A or G (purines)
Y: Represents C or T (pyrimidines)
S: Represents G or C
W: Represents A or T
K: Represents G or T

For example, any one or more of SEQ ID NOs. 1-56 may include one or more degenerate bases.

In some embodiments, a reaction within a reaction receptacle (e.g., well of a plate, well of a 96-well plate, a PCR tube (e.g., 0.1 mL, 0.2 mL, etc.)) may be refined for a concentration of one or more primers or a plurality of primers, a magnesium chloride (MgCl₂) concentration, a deoxynucleotide triphosphate (dNTP) concentration, a type of polymerase (e.g., Taq polymerase, DNA polymerase, etc.), a performance of a polymerase (e.g., Taq polymerase, DNA polymerase, etc.), a buffer composition, thermocycling conditions (e.g., initial denaturation, annealing temperature, extension time, isothermal conditions), and the like. Concentrations may be refined to balance the total final number of amplicons for a target locus within a pool for regions that may be amplified less-preferentially. As described above, in one non-limiting example, plant specific and fungi specific primers, when mixed together, may be in a 20:1 ratio, a 15:1 ratio, a 10:1 ratio, or a 5:1 ratio (plant primers to fungi primers) since fungi loci may be more preferentially amplified.

The kits and methods described herein may have various practical applications. For example, the kits and methods described herein may be practically applied for biosurveillance and/or biomonitoring, for example to detail, track, record, observe, or otherwise monitor trends in lifeforms over time or recent or current trends versus historical trends in lifeforms over time. Biosurveillance or biomonitoring performed using the kits and methods described herein, may be used to identify emerging species, disappearing species, or substantially stable species. In some embodiments, the kits and methods described herein may be used to generate suggested actions that may be taken to cultivate or support disappearing lifeforms (e.g., increase nutrients, increase access to food sources, reduce harvesting of the lifeform, etc.), mitigate or reduce invasive species (e.g., treat an area, reduce nutrients for the lifeform, introduce competing lifeforms, etc.), support or cultivate emerging lifeforms (e.g., increase nutrients, increase access to food sources, reduce harvesting of the lifeform, etc.), study emerging species (e.g., phenotypic studies, biological studies, genetic studies, etc.), etc. In some embodiments, biosurveillance or biomonitoring may include tracking threatened and/or endangered species. In some embodiments, biosurveillance or biomonitoring may include tracking invasive and/or culturally important species. In some embodiments, biosurveillance or biomonitoring may include detecting cryptic species. In some embodiments, biosurveillance or biomonitoring may include informing, adjusting, or otherwise impacting forest, wildlife and/or fishery management. In some embodiments, biosurveillance or biomonitoring may include habitat health monitoring. In some embodiments, biosurveillance or biomonitoring may include tracking species ranges due to climate change.

In some embodiments, the kits and methods described herein may be practically applied for monitoring, detecting, etc. environmental pathogens and/or human pathogens. The kits and methods described herein may be practically applied for monitoring indoor environments for lifeforms that may impact human health and/or wellness. For example, the kits and methods described herein may be used to monitor for potentially detrimental lifeforms, for example molds, pests, hospital acquired infections, community acquired infections, and the like. Various actions may be taken to remediate or remove (e.g., cleaning, fumigation, renovation, etc.) the detrimental lifeforms or provide therapies (e.g., asthma treatments, allergy treatments, etc.) to those impacted by the detrimental lifeforms. The kits and methods described herein may be practically applied for consumer use, at home use, and the like to track, monitor, record, etc. lifeforms that may impact human health in a work context, home context, school context, medical context, and the like. Various actions may be suggested based on results obtained from the kits and methods described herein to remediate or remove (e.g., cleaning, fumigation, renovation, etc.) the detrimental lifeforms or provide therapies (e.g., asthma treatments, allergy treatments, etc.) to those impacted by the detrimental lifeforms.

In some embodiments, the kits and methods described herein may be practically applied for forensic analysis. The kits and methods described herein may be used to assess a geographic origin of a sample based on a biodiversity present in the sample. For example, a lifeform associated with an article under investigation (e.g., blanket, article of clothing, vehicle, etc.) may be assessed to determine which lifeforms are associated with it and where those lifeforms are frequently found.

In some embodiments, the kits and methods described herein may be practically applied for monitoring agricultural pathogens. Various actions may be taken to alter an impact of one or more agricultural pathogens. For example, pesticide usage may be altered, complementary crops (e.g., three sisters crops-beans, corn, and squash) may be planted in the vicinity to support growth of the crop under analysis, additional litmus tests may be implemented (e.g., planting of roses near grape vines as a litmus test for grape vine health), etc.

In some embodiments, the kits and methods described herein may be practically applied for assessing environmental impacts, for example of new construction, drain fields, chemical plants, renewable energy sites, oil and gas development, etc. Various actions may be taken to alter an environmental impact. For example, replacement environments (e.g., wetlands, trees, etc.) may be incorporated into the design or otherwise planted or developed elsewhere to reduce impact. For example, an architecture of a design may be adjusted to include additional environmental features (e.g., rooftop green space, wetland on the property, etc.) to reduce environmental impact.

In some embodiments, the kits and methods described herein may be practically applied for monitoring a pollution response. Pollution over time, historical pollution versus current pollution trends, pollution changes as a result of new factories or industries being introduced in an area, impact of pollution over time on a biological community, etc. may be monitored. Various actions may be taken to alter a pollution impact, such as increase pollution reversal measures (e.g., planting more trees, increasing filters in buildings and factory outputs, altering water source usage by factories, etc.). In some embodiments, monitoring a pollution response may include identifying impacts of industry on an environment.

In some embodiments, the kits and methods described herein may be practically applied for ascertaining restoration success. An environment may be monitored over time using the kits and methods described herein to determine whether remediation efforts or other preservation efforts have improved an environment or returned an environment to a previous state or a reference state (e.g., a reference state in proximity to the target site), for example that was less impacted.

In some embodiments, the kits and methods described herein may be practically applied for monitoring global climate change effects. Impacts of weather changes, temperature changes, etc. and their impact on lifeforms may be monitored over time or historically versus current trends. Various actions may be taken to positively impact detrimental effects, for example, increasing resources for one or more lifeforms, decreasing negative impacts on one or more lifeforms, etc.

As used herein, a “lifeform” may be a living entity or being that contains DNA, for example a fungus, a bacterium, a virus, a mammal, a prokaryote, a eukaryote, a protist, a plant, an archaebacteria, an animal, a unicellular organism, a multicellular organism, a fish (e.g., cartilaginous or bony), a crustacean, etc.

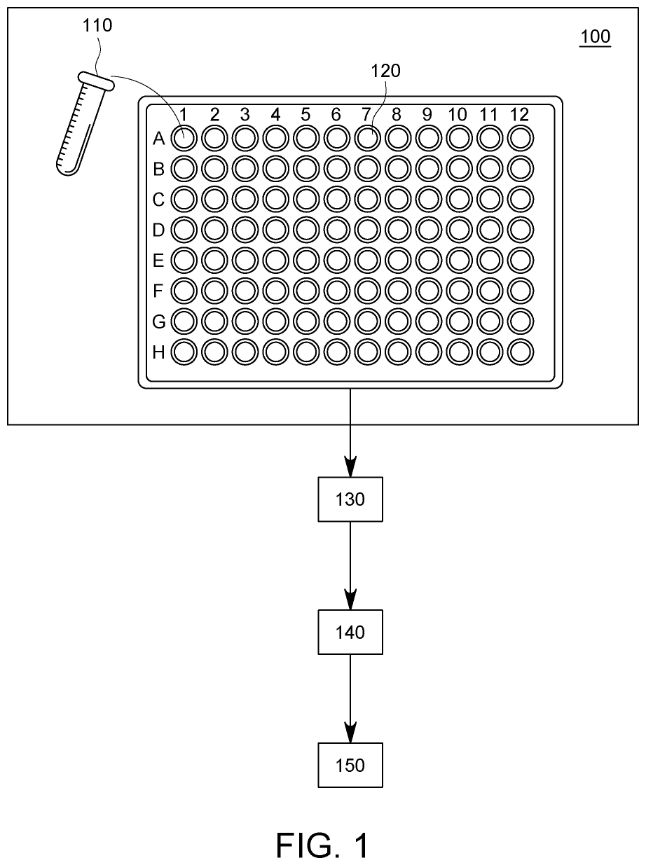

FIG. 1 shows a kit 100 for determining a biodiversity of an environmental sample. The kit 100 may include primers 110 disposed in one or more reaction receptacles 120 (e.g., a well of a 96-well plate). Alternatively, the primers 110 may be provided separately in the kit 100 such that the primers 110 may be added to the one or more reaction receptacles 120 upon receipt of the kit 100 or when the kit 100 is going to be used or planned to be used. The primer sets may be complementary to a taxonomic barcode region of a genome (i.e., a sequence of DNA that is taxonomically identifiable or the sequence of DNA is associated with a particular taxonomy). Said another way, the primer sets may be complementary to a target region that includes taxonomically important information about a lifeform. A taxonomically informative region, a taxonomically important region, or a taxonomic barcode region may be any portion of a target gene or target sequence (e.g., noncoding) that is amplified using the kits and methods described herein. The target region of DNA in the lifeform does not have to be a “barcode” region but can be a barcode region because that is where taxonomically identifiable reference data exists to process or demultiplex amplicon data. A primer or primer set, as shown in FIG. 3A, may include a first sequence including a tag sequence that can be demultiplexed to identify each environmental sample across the reaction receptacles (e.g., across wells of a multi-well plate). Optionally, a primer or primer set may include an adapter sequence for a particular sequencing technology (e.g., Illumina®), as shown in FIG. 3C. In some embodiments, a primer set may include a plurality of primers including one or more forward primers and one or more reverse primers, such that the plurality of primers does not form obligate 1:1 pairs of forward and reverse primers. A primer or primer set may include a taxonomically informative sequence (e.g., a phylogenetically targeted sequence that may be used to amplify one or more specific narrow or broad taxonomic groups) that is complementary to a region of DNA of a taxonomic group. The taxonomically informative sequence may be demultiplexed, as described elsewhere herein, to identify whether the taxonomic group is present in one or more environmental samples.

In some embodiments, a first subset of primers may include a first taxonomically informative sequence that is complementary to a first region of DNA of a first taxonomic group. In some embodiments, a second subset of primers may include a second taxonomically informative sequence that is complementary to a second region of DNA of a second taxonomic group. In some embodiments, the first taxonomically informative sequence and the second taxonomically informative sequence may be demultiplexed to identify whether one or both of the first taxonomic group and the second taxonomic group are present in one or more environmental samples. In some embodiments, one or more lifeforms or species may belong to both a first taxonomic group and a second taxonomic group. In some embodiments, one or more lifeforms may belong to a first taxonomic group and not a second taxonomic group (or vice versa).

A sample may be added to each reaction receptacle 120 so that DNA from the sample can be amplified by a PCR method 130. The sample may be a raw sample (i.e., unmanipulated). Alternatively, the sample may be extracted DNA from a sample. The sample may be reverse transcribed RNA (that was isolated from an environmental sample) to complementary DNA (cDNA). The sample may be a single cell sample. Alternatively, or additionally, the sample may be fragmented, extracted DNA from a sample. Further still alternatively, or additionally, the sample may be circularized DNA from a sample such that PCR method 130 is a rolling circle amplification (RCA) method. The resulting amplicons from PCR method 130 may be about 50 base pairs to about 350 base pairs, about 50 base pairs to about 500 base pairs, about 300 base pairs to about 500 base pairs, about 500 base pairs to about 1,000 base pairs, about 500 base pairs to about 5000 base pairs, etc.

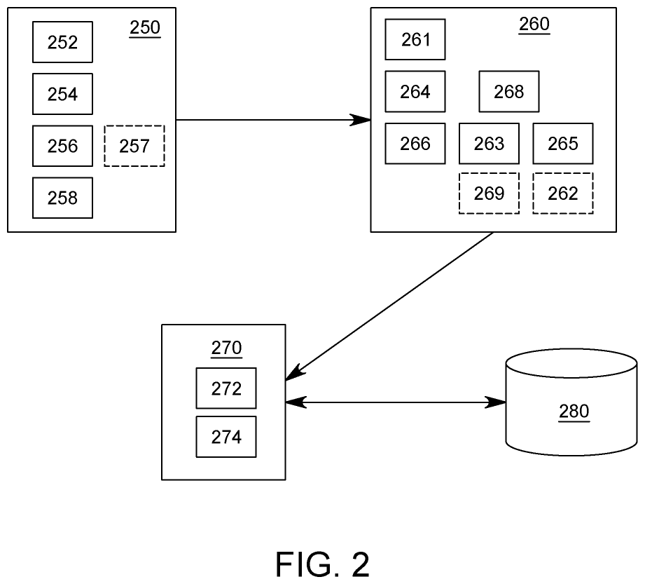

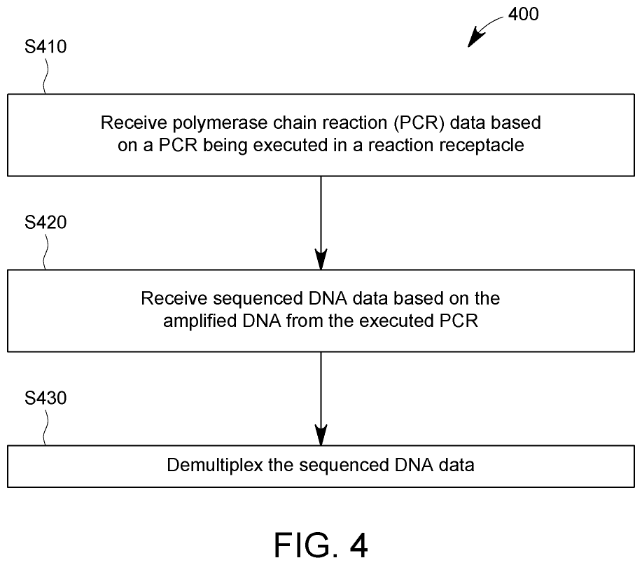

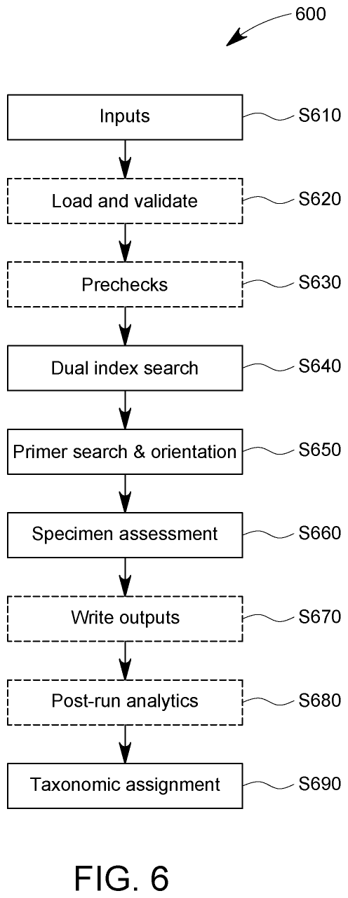

The amplicons from the PCR method 130 may be sequenced using sequencing method 140. The sequencing method may use the DNA sequencer 260, or similar sequencer, as described elsewhere herein and at least with respect to FIG. 2. The DNA sequencing data may be processed at processing step 150. For example, the DNA sequencing data may be demultiplexed, as described elsewhere herein and at least with respect to FIG. 4 and FIG. 6. The DNA sequencing data may be used to calculate a biodiversity score or credit, as described elsewhere herein. The DNA sequencing data may be processed to determine a taxonomic assignment or annotation of the environmental sample. The DNA sequencing data may be processed to determine a number of species or taxonomies present in the environmental sample, as described with respect to FIG. 4. The processed DNA sequencing data may result in an output of a taxonomic assignment (e.g., assigned species, assigned, genus, assigned class, assigned order, assigned family, assigned kingdom, assigned phyla, etc.) for one or more samples. For example, for a sample, a first taxonomic group may encompass plants, and the second taxonomic group may encompass bacteria. For a sample, the first taxonomic group may encompass plants, and the second taxonomic group may encompass bacteria. For a sample, the first taxonomic group may encompass fungus, and the second taxonomic group may encompass bacteria. For a sample, the first taxonomic group may encompass fungus, and the second taxonomic group may encompass plants. For a sample, the first taxonomic group may encompass a first subset of fungal species, and the second taxonomic group may encompass a second subset of fungal species. In some embodiments, there may be overlap in species between the first subset of species and the second subset of species. Although various taxonomic groupings and assignments and combinations are described above, one of skill in the art will appreciate that these groupings, assignments, and combinations are merely illustrative and should not be construed as limiting.

Further, for example, processing at block 150 may include base calling (e.g., using trained models or statistical techniques). Base calling includes identifying a nucleotide sequence from the output data produced during DNA sequencing. After sequencing, the DNA sequencer 260, or an electrically coupled computing device 270, outputs sequencing data (e.g., light signals, voltage changes, electrical signals depending on the technology used, e.g., Illumina®, Oxford Nanopore®, PacBio®). Base calling includes determining the specific bases (A, T, G, C) in the DNA strand based on the sequencing data. For example, base calling may include extracting a pattern from the output sequencing data to assign each signal to a nucleotide.

In some embodiments, processing at block 150 may include quality filtering. Quality filtering may include removing low-quality reads or nucleotides to increase the reliability of downstream analyses. A base call may be assigned a quality score (e.g., Phred scores) indicating a probability that the base call is correct. As a non-limiting example, a Phred score (Q) is logarithmically related to the error probability (P), using the formula: Q=−10 log₁₀P. Higher scores mean greater confidence in the base call (e.g., a Phred score of 30 means a 1 in 1000 chance of an error).

In some embodiments, processing at block 150 may include base trimming. Low-quality bases (e.g., bases with a Phred scores below a predefined threshold) may be trimmed from the ends of reads. Sequencing quality tends to degrade toward the ends, for example in technologies like Illumina®. Trimming poor-quality ends improves the overall quality of reads. In some embodiments, reads that are too short (e.g., below a predefined length threshold and/or after trimming) may be removed. In some embodiments, reads that have an average quality score below a predefined threshold may be filtered out.

In some embodiments, processing at block 150 may include removing sequencing adapters. Sequencing adapters, which may not have been removed during the sequencing process, may be identified and removed. Adapters are non-biological sequences that can introduce errors in downstream analysis if not removed.

In some embodiments, processing at block 150 may further include outputting a sequence of nucleotides in a digital format (e.g., FASTQ), which may optionally include the sequence information and the quality scores for one or more bases.

In some embodiments, processing at block 150 may optionally include clustering. Clustering after DNA sequencing may include aggregating similar DNA sequences together to reduce data complexity and/or enhance analysis efficiency. Clustering may include sequence dereplication which includes combining identical sequences and recording a frequency of the identical sequences. Clustering may include generating a pairwise distance matrix by comparing the sequences. The distance between sequences may be defined based on the number of mismatches (or similarity percentage) between the sequences. A lower distance indicates greater similarity between two sequences. One or more clustering algorithms may be used to group sequences based on their similarity. For example, in greedy clustering, sequences are sequentially added to clusters, starting with the most abundant sequence as the representative of the first cluster. Similar sequences within a specified similarity threshold (e.g., 97% for species-level clustering) may be grouped into the same cluster. For example, in hierarchical clustering, a tree-like structure may be generated that groups sequences based on their similarity. Hierarchical methods can be agglomerative (bottom-up) or divisive (top-down). For example, in heuristic approaches, tools (e.g., UCLUST, VSEARCH) may use heuristic methods to identify clusters without having to compare sequence pairs. In operational taxonomic units (OTU) identification, sequences are grouped into OTUs based on a predefined similarity threshold (e.g., 97%, 98%, 99%, etc.) to approximate species-level classification. OTUs may be used as an estimate or proxy for taxonomic assignments. In some embodiments, clustering may include identifying Amplicon Sequence Variants (ASVs). ASVs represent distinct sequences without a predefined similarity threshold, resulting in higher resolution than OTUs. In cluster representatives, a representative sequence is selected (e.g., an abundant sequence) to represent a cluster in a downstream analysis. These representative sequences can be used for taxonomic classification or creating phylogenetic trees.

FIG. 2 shows various devices or systems that may execute one or more methods or portions of methods described elsewhere herein. One or more devices for executing the kits and methods for biodiversity recovery in a sample may include, but not be limited to, a thermal cycler 250 for polymerase chain reactions (PCR), a DNA sequencer 260, a computing device 270 (e.g., workstation, quantum computer, laptop, mobile computing device, etc.) for data analysis and/or signal or data demultiplexing, and optional database 280. The one or more devices may be used for amplifying a plurality of target sequences (e.g., using thermal cycler 250), sequencing a plurality of output nucleic acids (i.e., amplicons) from the thermal cycler 250 (e.g., using DNA sequencer 26), and/or analyzing one or more sequenced nucleic acids (e.g., using computing device 270) optionally using or referring to database 280.

The thermal cycler 250 may control a temperature of one or more reactions to facilitate the enzymatic reactions (e.g., polymerase reactions) for DNA amplification. Although thermal cycler 250 is described as a “cycler” herein, one of skill in the art will appreciate that thermal cycler 250 may also be used for isothermal amplification reactions such as Loop-Mediated Isothermal Amplification (LAMP) or RCA. Thermal cycler 250 may include a processor 252 (e.g., microcontroller, digital signal processor (DSP), field-programmable gate array (FPGA), etc. integrated or external), a heating/cooling system 254 (e.g., Peltier element), an optional optical detection system 257 (e.g., for real-time PCR), one or more sensors 256 (e.g., temperature sensor), and a power source 258 (e.g., electrically connected to an outlet or including a battery or other power source). The heating/cooling system 254 may control a heating and cooling of a thermal block in which the reaction receptacles reside to achieve the various steps of the PCR reaction including denaturation, annealing, and extension. The processor 252 may control one or more temperature cycles and/or hold times during a PCR cycle. The processor 252 may receive one or more inputs indicating a desired parameter, such as temperatures and/or duration of a step in the PCR reaction or a number of cycles. The processor 252 may receive the one or more inputs through an optional graphical user interface on the thermal cycler 250 or using an electrically coupled computing device. The optical detection system 257 may include one or more light sources (e.g., laser, light-emitting diodes (LED)) and detectors (e.g., photodiodes, photomultiplier tubes, charge-coupled devices (CCD), spectrophotometers, etc.) for causing and detecting, respectively, fluorescence signals from one or more samples and/or reaction receptacles during a PCR cycle. The PCR reactions may be quantitative PCR (qPCR) reactions or standard PCR.

DNA sequencer 260 may be used to determine a sequence of nucleotides, adenine (A), thymine (T), cytosine (C), and guanine (G), in a DNA molecule. DNA Sequencer 260 may include a fluidics system 264, a thermal control system 266, an image and signal processing system 268, a processor 263 (e.g., central processing unit (CPU), graphics processing unit (GPU), DSP, FGPA, application-specific integrated circuit (ASIC), microcontroller, neural processing unit etc. external to the sequencer 260 or integrated into the sequencer 260), a data analysis system 265, a power source 261, and an optional nanopore system 269 (e.g., for nanopore sequencing methods), an optional optical detection system 262 (e.g., for Illumina® sequencing methods), or another sequencing technology. The fluidics system 264 may function to supplies enzymes, nucleotides, and/or buffers for the sequencing reactions. The fluidics system 264 may optionally include one or more channels or wells for immobilizing DNA fragments for sequencing. The thermal control system 266 may maintain or adjust to various temperatures during the sequencing reactions. The image and signal processing system 268 may include signal amplifiers for processing raw fluorescence or electrical signals generated during sequencing. The image and signal processing system 268 may include a data converter for converting analog signals into digital data for base calling (i.e., determining the nucleotide sequence). In some embodiments of Illumina® sequencers, the image and signal processing system 268 may include an image processor for generating images of fluorescent signals to determine the sequence. The data analysis system 265 may include base calling software to interpret the signals (e.g., fluorescence signals, electrical signals, etc.) to identify a nucleotide sequence of a DNA molecule. The data analysis system 265 may include an alignment software to align sequences to reference genomes (e.g., in database 280) or may include de novo assembly algorithms. The data analysis system 265 may include an error checking algorithm to identify errors in the sequencing data and output quality scores. The data analysis system 265 may include one or more bioinformatics tools or algorithms to analyze the sequence data to identify mutations, variants, and/or structural rearrangements in the DNA. The DNA sequencer 260 may optionally include a user interface to receive one or more user inputs to adjust a DNA sequencing method or step, input sequencing parameters (e.g., run length, reagent volumes, temperature settings, etc.), and the like. DNA sequencer 260 may optionally include a graphical user interface or command-line interface to monitor the sequencing process and visualize the output. The power source 261 may be an electrically connected outlet, or the DNA sequencer 260 may include a battery or other power source. The optional nanopore system 269 (e.g., in nanopore sequencers) may include one or more nanopores or channels in the flow cell that have their electrodes connected to a sensor chip which quantifies electric current variation induced by different nucleotides traversing the nanopore with membrane embedding. The DNA molecules pass through the nanopores or channels, which causes changes in electrical conductivity to provide data on the nucleotide sequence (i.e., differentiates different types of nucleotides, such as A, T, G, and C). Said another way, the nanopore system 269 may include a membrane and electrodes to detect changes in ionic current as the DNA passes through one or more pores or channels. The optional optical detection system 262 (e.g., in Illumina® sequencers) may include one or more light sources (e.g., laser, LED, etc.) to excite fluorescently labeled nucleotides or dyes attached to the DNA fragments during sequencing. The optical detection system 262 may further include one or more detectors (e.g., CCD, complementary metal-oxide semiconductor (CMOS), etc.) to capture the emitted fluorescence from each nucleotide as it is added during sequencing. The intensity and color of the fluorescence correspond to specific nucleotide bases. The optical detection system 262 may further include one or more filters to select specific wavelength or one or more wavelengths of light emitted from the fluorophores to distinguish between different nucleotide bases. Other sequencing technologies may also be used, in addition to, or alternatively to Illumina® and Nanopore®, for example Element Aviti®, Complete Genomics, etc.

The computing device 270 may be electrically coupled (e.g., databus) or otherwise communicatively coupled (e.g., Zigbee, Wi-Fi, other wireless protocol, etc.) to the thermal cycler 250 and/or DNA sequencer 260. Alternatively, data may be moved manually between the computing device 270 and the thermal cycler and/or DNA sequencer 260. In some embodiments, the computing device 270 is integrated with the thermal cycler 250, such that the computing device 270 that is used with the thermal cycler 250 may also be used for data analysis. In some embodiments, the computing device 270 is integrated with the DNA sequencer 260, such that the computing device 270 that is used with the DNA sequencer 260 may also be used for data analysis. In some embodiments, the computing device 270 is a standalone machine or workstation or a remote computing device. The computing device 270 may include a processor 272 and memory 274, such that the memory 274 stores computer readable instruction that, when executed by the processor 272, cause the processor 272 to execute one or more data analysis algorithms, one or more methods for determining a biodiversity of an environmental sample, and the like, as described elsewhere herein and as described with respect to FIGS. 1 and 3-5. Computing device 270 may execute one or more data analysis algorithms, described elsewhere herein, for example one or more base calling algorithms, one or more quality filtering algorithms, one or more base trimming algorithms, one or more adapter sequence removal algorithms, one or more clustering algorithms, and/or one or more data demultiplexing algorithms. One or more demultiplexing algorithms are described with respect to FIG. 4 and FIG. 6 and elsewhere herein.

Computing device 270 may be communicatively coupled to optional database 280 (e.g., local database, remote database, or database stored in the Cloud). Database 280 may store a plurality of reference sequences of lifeforms. The reference sequences may be compared to one or more output sequences of the methods described herein to determine a taxonomy present in a sample, a species present in a sample, a lifeform present in a sample, a biodiversity present in a sample, and the like.

Based on the comparison or another analysis method, the computing device 270 may determine a biodiversity score. Metrics of biodiversity may include, but not be limited to, species richness, species evenness, functional diversity, genetic diversity, keystone species presence, habitat heterogeneity, sample heterogeneity, endemic species, rare species, spatial connectivity, etc. Species richness (e.g., species richness=number of distinct species in an area or sample) may be described as the number of different species present in a given area or ecosystem. For example, species richness may include defining an area or sampling unit (e.g., 1 m² quadrat, 1 km² region, or a river stretch); and determining a number of unique species in the area or sampling unit using any of the methods described herein or generally known to one of skill in the art.

Species richness may be a measure of biodiversity and may indicate the variety of species without considering their abundances. For example, species richness may be determined by determining whether species richness should be assessed temporally versus spatially; determining a number of unique species in an area or over time using any of the methods described herein or generally known to one of skill in the art; and calculating a species richness based on the following exemplary, non-limiting formula:

$$S = {{\sum }_{i = 1}}^{n} {I}_{i}$$

where:
n=total number of possible species in the dataset
Iᵢ=1 if species i is present, 0 if otherwise.

Species richness, for example as part of a biodiversity metric, may be compared across sites, adjusted for sampling effort (e.g., using rarefaction curves or Chao1 estimator), and/or normalized to create a unitless index between zero and one for comparison.

Species evenness may be defined as similar abundance of a species across samples or similar richness of species across samples. Species evenness may be determined using Shannon diversity (H′) or Simpson's diversity (D) or other methodologies known to one of skill in the art.

Genetic diversity may be described as the variety of genetic material within a species or population. Genetic diversity may include the differences in genes and alleles among individuals, providing the basis for adaptability and evolution. For example, unique alleles at one or more or each genetic locus may be determined and standardized for sample size using rarefaction. Further, for example, observed heterozygosity may be determined by measuring a proportion of individuals carrying two different alleles at a locus. Further measures of genetic diversity may include, but not be limited to expected heterozygosity calculations, nucleotide diversity calculations, fixation index calculations, and the like.

Keystone species presence may be described as an existence of species in an ecosystem that have a disproportionately large effect on their environment relative to their abundance. Keystone species play particular roles in maintaining the structure and function of ecosystems. Habitat heterogeneity may be described as the variety and complexity of physical environments within an ecosystem. Habitat heterogeneity may include different habitat types, structures, and resources, which support a diversity of species and ecological interactions. Sample heterogeneity may be described as the variation in species composition or other ecological factors across different samples or locations within an area. Sample heterogeneity may reflect differences in biodiversity and community structure within the studied region. Endemic species may be described as species that are native to and found only within particular geographical regions. Endemic species have restricted ranges, making them unique to their area of origin and often vulnerable to extinction. Rare Species may be described as species that have small population sizes, limited distributions, or both. Rare species may be at higher risk of extinction due to their low numbers or restricted habitats. Spatial Connectivity may be described as the degree to which different habitats or populations are connected within a landscape. High spatial connectivity allows for movement and dispersal of species, supporting genetic diversity, species migration, and ecosystem resilience.

In some embodiments, a biodiversity score may be determined by comparing a historical biodiversity and/or phylogenetic diversity with a current biodiversity and/or phylogenetic diversity. Based on the comparison or another analysis method, the computing device 270 may determine a biodiversity score, for example, by comparing a reference area with known high or low levels of biodiversity and/or phylogenetic diversity with a current biodiversity and/or phylogenetic diversity. Based on the comparison or another analysis method, the computing device 270 may determine a biodiversity score, for example, by comparing a biodiversity for similar samples, similar environments, similar geographic regions, etc. to a current sample undergoing analysis. When the biodiversity in a prior sample substantially equals a biodiversity in a current sample, controlled for outlying differences, the biodiversity score may be around a predefined threshold. For example, the biodiversity score may be about 100% or a similar value or indicator meaning that the biodiversity is as expected. When the biodiversity in a prior sample is substantially less or negatively different than a biodiversity in a current sample, controlled for outlying differences, the biodiversity score may be less than a predefined threshold. For example, the biodiversity score may be less than about 100% or a similar value or indicator meaning that the biodiversity is less than expected. When the biodiversity in a prior sample is substantially greater than or positively different than a biodiversity in a current sample, controlled for outlying differences, the biodiversity score may be greater than a predefined threshold. For example, the biodiversity score may be greater than about 100% or a similar value or indicator meaning that the biodiversity is greater than expected. In some embodiments, calculating a biodiversity score may include weighting the calculation based on a type of environment in which the sample was found (e.g., endangered environment, plentiful environment, at risk environment, ocean environment, agricultural environment, arid environment, tropical environment, urban environment, rural environment, etc.), a type of expected impact that is being investigated (e.g., pollution, new factory or industry site, etc.), a geographic location from which the sample was taken (e.g., North America, Antarctica, Sub-Saharan Africa, etc.), a type of sample, a quality of the sample, quantity of samples across a given area, a percent of the expected total biodiversity that was captured, etc. In some embodiments, the biodiversity score may include a taxonomic assignment of at least one environmental sample.

In some embodiments, a biodiversity score may be used to calculate or determine a biodiversity credit. For example, a biodiversity credit may represent a measurement of ecological uplift or an increase in measurable biodiversity as a result of conservation or restoration, in some embodiments. A biodiversity credit may indicate substantial maintenance (e.g., relative to baseline, to an equivalent location or site, etc.) of a biodiversity at a location. A biodiversity credit may represent a substantial increase or a biologically healthy increase (e.g., percent increase, increase above a predefined threshold, etc.) in a biodiversity at a location. Determining a biodiversity credit may include determining a baseline biodiversity or an amount of biodiversity that would have occurred without any intervention. This baseline may be used as a reference point to measure biodiversity improvements or reductions, for example as a result of positive or negative interventions, respectively. A biodiversity score may be monitored and/or measured over a predefined time period to further be fed into or used to calculate the biodiversity credit. Further, biodiversity reductions or improvements may be calculated by comparing the baseline biodiversity with the actual biodiversity after implementing the project or after the impact (e.g., new infrastructure, new land use, etc.). The difference between these values may represent the degree to which the biodiversity was impacted.

A computer-implemented method for calculating biodiversity credits may include receiving, by a computing system, a baseline biodiversity or biodiversity score for a defined land area, the baseline biodiversity or biodiversity score. The biodiversity or biodiversity score may include species presence or abundance data based on the systems and methods described herein. The method may include receiving, by the computing system, a post-intervention biodiversity or biodiversity score for the defined land area following a restoration or conservation activity. The method may include computing, by the computing system, a baseline biodiversity index (BBI) and a post-intervention biodiversity index (PBI) using a biodiversity valuation model that incorporates, for example, biodiversity data, a biodiversity score, a species richness, a habitat quality, an ecological connectivity parameter(s), and the like. The method may include determining, by the computing system, a biodiversity uplift value (BUV) according to the PBI minus the BBI multiplied by the defined land area. The BUV may be converted into a biodiversity credit quantity (BCQ) based on a predefined conversion factor. Biodiversity credits may be output to a registry or database or stored in a distributed ledger. The biodiversity credit may be stored with associated metadata, for example, including project identifier, geolocation, verification timestamp, and data provenance indicators.

Generating, by the computing system, a digital record representing a tradable biodiversity credit token corresponding to the biodiversity credit quantity.

As shown in FIG. 4, a computer-implemented method 400 for determining a biodiversity of an environmental sample of an embodiment includes receiving PCR amplicons based on a PCR being executed in a reaction receptacle at block S410, receiving sequenced DNA data based on the amplified DNA from the executed PCR at block S420, and demultiplexing the sequenced DNA data at block S430. The method 400 functions to determine a number and/or type of lifeforms in a sample, for example an environmental sample. In some embodiments, the method functions to calculate a biodiversity score or credit, as described else wherein herein. The method can be used for biosurveillance, biomonitoring, forensics, environmental impact, agricultural monitoring, invasive species monitoring, pollution monitoring, restoration monitoring, global climate change monitoring, forest management, wildlife management, fishery management, habitat monitoring, industry impact monitoring, pathogen monitoring, etc., but can additionally, or alternatively, be used for any suitable applications, clinical, medical, environmental, or otherwise. The method can be adapted to function for any suitable field or industry in which lifeform assessment, monitoring, impact, etc. may be useful.

At least portions of the method 400 may be performed by computing device 270, as a standalone computing device or integrated with one or more of: the thermal cycler 250 and/or the DNA sequencer 260. In some embodiments, the method may be performed by another computing device or a remote computing device. The method 400 may be performed by a processor 272 of computing device 270 or one or more processors 272 of one or more computing devices 270. The method 400 may be performed by a processor 252 of thermal cycler 250 or one or more processors 252 of one or more thermal cyclers 250. The method 400 may be performed by a processor 263 of DNA sequencer 260 or one or more processors 263 of one or more DNA sequencers 260. The method 400 may be performed by a processor or one or more processors of another computing device(s) or a remote computing device(s).

As shown in FIG. 4, an embodiment of a computer-implemented method 400 for determining a biodiversity of an environmental sample includes block S410, which recites receiving PCR amplicons based on a PCR being executed in a reaction receptacle. An environmental sample may be added to each reaction receptacle. For example, the thermal cycler 250 of FIG. 2 or a similar device may be used to amplify DNA sequences using one or more PCR reactions in a reaction receptacle. The reaction receptacle may be a multi-well plate, for example a 24-well plate, a 96-well plate, a 384-well plate, etc. such that the reaction receptacle includes a plurality of reaction receptacles. PCR amplicons may be generated for each reaction receptacle or each well. The PCR amplicons (e.g., PCR or quantitative PCR) may be generated from one or more PCR reactions, amplicons purified from gel electrophoresis, and the like. Each reaction receptacle may include a plurality of primers, or a primer set and buffers, enzymes, nucleotides, etc. Each primer may be complementary to at least one taxonomic barcode region of a genome of a lifeform.

FIGS. 3A-3C show various PCR methodologies that may be used with the kits and methods described herein. In a single step PCR process 300, as shown in FIG. 3A, a plurality of primers 304, 306, 314, 318 may be present in the reaction. Each primer may include a tag sequence to identify each sample or each reaction receptacle, for example relative to a plurality of other samples or a plurality of other reaction receptacles. For example, primers 304, 306, 314, 318 each have sample tag sequence 302. Primers 304, 306 bind to the 5′ to 3′ strand 308 of the double stranded DNA and primers 314, 318 bind to the 3′ to 5′ strand 310 of double stranded DNA. The primers 304, 306, 314, 318 amplify the DNA n number of times at arrow 322. The resulting amplicons 324, 326, 328, 330 are shown. Amplicon 324 is the amplification product of primer 306 and primer 314. Amplicon 326 is the amplification product of primer 304 and primer 314. Amplicon 328 is the amplification product of primer 306 and primer 318. Amplicon 330 is the amplification product of primer 304 and primer 318. These amplicon permutations may be compared to a library to determine which primers amplified or generated each amplicon—in other words to demultiplex the permutations. The library, for example stored in database 280, may include a lookup table of various primer combinations based on the plurality of primers not forming obligate 1:1 pairs. Comparing the amplicon permutations to the library may identify which primers were used to generate which amplicons to demultiplex the permutations to identify which specie(s) were present in the sample or reaction receptacle.

In a dual step PCR reaction, as shown in the processes in FIGS. 3B-3C, a first PCR reaction 350 (FIG. 3B) may add linker sequences for DNA sequencing and a second reaction 380 (FIG. 3C) may add tags for sample ID. In the first PCR reaction 350, as shown in FIG. 3B, a plurality of primers 354, 356, 358, 360 may be present in the reaction. Each primer may include a linker sequence 352 to facilitate DNA sequencing. For example, primers 354, 356, 358, 360 each have a linker sequence 352. Primers 354, 356 bind to the 5′ to 3′ strand 372 of the double stranded DNA and primers 358, 360 bind to the 3′ to 5′ strand 374 of the double stranded DNA. The primers 354, 356, 358, 360 amplify the DNA n number of times at arrow 362. The resulting amplicons 364, 366, 368, 370 are shown. Amplicon 364 is the amplification product of primer 356 and primer 358. Amplicon 366 is the amplification product of primer 354 and primer 358. Amplicon 368 is the amplification product of primer 356 and primer 360. Amplicon 370 is the amplification product of primer 354 and primer 360. These amplicon permutations may be used in a second PCR reaction 380. In the second PCR reaction 380, as shown in FIG. 3C, a plurality of primers having the linking sequence 352 and a tag sequence 382 for sample ID may be present in the reaction. The linker sequence 352 of the primers in FIG. 3C is complementary to the linker sequence 352 in the amplicons 364, 366, 368, 370 from the first PCR reaction 350. Amplification of the amplicons 364, 366, 368, 370 in the second PCR reaction at arrow 390 results in amplicons 388, 392, 394, 396 that are the amplicons 364, 366, 368, 370, having a linker sequence 352 and a tag sequence 382. These amplicons 388, 392, 394, 396 permutations may be compared to a library to determine which primers amplified each amplicon—in other words to demultiplex the permutations, as described above.

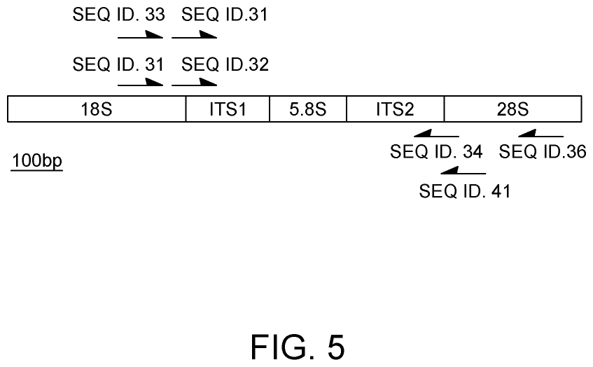

Further, as shown in FIG. 5, each primer set may include a plurality of primers that do not form obligate 1:1 pairs. For example, as shown in FIG. 5, there are a plurality of primers (e.g., in this example seven primers) complementary to the nrITS operon for fungi that may amplify a plurality of intervening sequences (e.g., twelve permutations in this example), depending on which primers are pairing together in a reaction. For example, forward primer SEQ ID NO. 33 may pair with reverse primer SEQ ID NO. 36 to amplify the intervening sequence. Forward primer SEQ ID NO. 33 may pair with reverse primer SEQ ID NO. 34 to amplify the intervening sequence. Forward primer SEQ ID NO. 33 may pair with reverse primer SEQ ID NO. 35 to amplify the intervening sequence. Forward primer SEQ ID NO. 31 may pair with reverse primer SEQ ID NO. 36 to amplify the intervening sequence. Forward primer SEQ ID NO. 31 may pair with reverse primer SEQ ID NO. 34 to amplify the intervening sequence. Forward primer SEQ ID NO. 31 may pair with reverse primer SEQ ID NO. 35 to amplify the intervening sequence. Forward primer SEQ ID NO. 31 may pair with reverse primer SEQ ID NO. 36 to amplify the intervening sequence. Forward primer SEQ ID NO. 31 may pair with reverse primer SEQ ID NO. 34 to amplify the intervening sequence. Forward primer SEQ ID NO. 31 may pair with reverse primer SEQ ID NO. 35 to amplify the intervening sequence. Forward primer SEQ ID NO. 31 may pair with reverse primer SEQ ID NO. 36 to amplify the intervening sequence. Forward primer SEQ ID NO. 31 may pair with reverse primer SEQ ID NO. 34 to amplify the intervening sequence. Forward primer SEQ ID NO. 31 may pair with reverse primer SEQ ID NO. 35 to amplify the intervening sequence. As described elsewhere herein, one or more of the primers may have one or more degenerate bases to facilitate primer annealing to the DNA.

In some embodiments, each primer may include a taxonomically informative sequence that is complementary to a region of taxonomically informative DNA. For example, the taxonomically informative sequence may be a sequence of DNA that is conserved or substantially conserved among lifeforms or organisms belonging to a particular taxonomy (e.g., species, genus, class, order, family, kingdom, phyla, etc.). The taxonomically informative sequence may be a sequence of DNA that is shared or substantially shared among lifeforms or organisms belonging to a particular taxonomy. The taxonomically informative sequence may be a sequence of DNA that encodes an end product (e.g., protein, short noncoding RNA, etc.) that is a conserved or substantially conserved among lifeforms or organisms belonging to a particular taxonomy. The taxonomically informative sequence may be used to identify at least a taxonomy of lifeforms that may be present in the sample. The taxonomically informative sequence may be used to identify one or more taxonomic groups of interest in the sample.

In some embodiments, a first subset plurality of primers includes a first taxonomically informative sequence that is complementary to a first region of DNA of a first taxonomic group, and a second subset of the plurality of primers includes a second taxonomically informative sequence that is complementary to a second region of DNA of a second taxonomic group. The first and second taxonomically informative sequences may be used to identify at least a first subset of lifeforms in the sample and a second subset of lifeforms in the sample, respectively. Although first and second taxonomically informative sequences are used herein, one of skill in the art will appreciate that any number of taxonomically informative sequences may be used, for example 2 to 3, 2 to 5, 3 to 5, 5 to 10, 2 to 10, 2 to 20, 15 to 20, 10 to 30, etc.

In non-limiting embodiments, the first taxonomic group may encompass plants, and the second taxonomic group may encompass bacteria. The first taxonomic group may encompass plants, and the second taxonomic group may encompass bacteria. The first taxonomic group may encompass fungus, and the second taxonomic group may encompass bacteria. The first taxonomic group may encompass fungus, and the second taxonomic group may encompass plants. The first taxonomic group may encompass a first subset of fungal species, and the second taxonomic group may encompass a second subset of fungal species. For example, there may be an overlap in species between the first subset of fungal species and the second subset of fungal species. In some embodiments, there may be an overlap in species between the first taxonomic group and the second taxonomic group.

In some embodiments of method 400, receiving PCR amplicons may include generating data based on PCR-generated amplicons ranging in size from about 50 base pairs to about 350 base pairs, about 50 base pairs to about 500 base pairs, about 350 base pairs to about 500 base pairs, about 500 base pairs to about 5000 base pairs, etc.

In some embodiments, method 400 may further include extracting DNA (e.g., phenol-chloroform, chromatography, etc.) from the environmental sample. The method 400 may further include fragmenting (e.g., mechanical, enzymatic, etc.) the extracted DNA sequences into shorter DNA sequences (e.g., about 100 base pairs to about 300 base pairs, about 100 base pairs to about 150 base pairs, about 150 base pairs to about 300 base pairs, about 200 base pairs to about 300 base pairs, etc.). The method 400 may further include ligating tags to the fragmented DNA sequences. Alternatively, tags may be added to the DNA sequences during PCR, such that the primers include tags. In some embodiments, the tagged DNA sequences may be amplified by PCR, as described elsewhere herein.

As shown in FIG. 4, an embodiment of a computer-implemented method 400 for determining a biodiversity of an environmental sample includes block S420, which recites receiving sequenced DNA data based on the amplified DNA from the executed PCR. The PCR-generated amplicons may be fed into a DNA sequencer, for example the DNA sequencer 260 as shown in FIG. 2. The DNA sequencer 260 reads the sequence of nucleotides of the input DNA, for example, outputting sequenced DNA data indicating a sequence of nucleotides (A, T, C, G) for a plurality of DNA molecule fed into the DNA sequencer 260. The DNA sequencing data may be pre-treated or otherwise quality controlled by one or more of: length filtering, quality score filtering, dereplication, removing primer sequences, removing adapter sequences, chimera filtering, or the like.

As shown in FIG. 4, an embodiment of a computer-implemented method 400 for determining a biodiversity of an environmental sample includes block S430, which recites demultiplexing the sequenced DNA data. The sequenced DNA data may be demultiplexed based on a sample tag sequence integrated into each primer set, which identifies from which sample the DNA originated. The sequenced DNA data may be demultiplexed based on each permutation of each possible primer combination, as described above, that is present in the primer pool (e.g., the primer set in a reaction receptacle). Said another way, the sequenced DNA data may be permutationally demultiplexed. Said still another way, the sequenced DNA data may be compared to the n number of permutations of the primers in the PCR reaction. In some embodiments, one or more of the demultiplexing methodologies of block S650 of method 600 may be used and/or applied to block S430 of FIG. 4. For example, associating a read with a tag, confirming orientation of a read, and/or trimming primer sequences from the read may be performed. Further for example, block S430 may include assigning a read to a primer-pair-specific bin using a primer lookup based on a primer pool context and determining which forward and reverse primers are valid for that subset of reads. Block S430 may include primer position validation and/or primer orientation determination.

In some embodiments, the DNA sequencing data may be further demultiplexed to determine a biodiversity in one or more samples. The demultiplexing to determine the biodiversity may be based on the primer set being specific to an individual barcode region of a taxonomy or one or more individual barcode regions of a taxonomy.

In some embodiments and as described elsewhere herein, the method 400 may further include comparing the sequenced DNA data to a reference database of reference sequences and outputting an indication of a taxonomic composition of at least one environmental sample based on the demultiplexing the sequenced DNA data and the comparing.

In some embodiments, each primer of the plurality of primers includes a unique molecular identifier (UMI) for sequencing, such that demultiplexing includes identifying the first taxonomic group and the second taxonomic group using each unique molecular identifier in the sequenced DNA data. In some embodiments, there may be a plurality of demultiplexing permutations (e.g., up to or about three). The sequencing data may be demultiplexed based on the sample tag sequence, the primer sequences, and sorting to cluster reads with a UMI. A UMI is a short, random sequence of nucleotides that is added to each DNA molecule in a sample before amplification (e.g., PCR). UMIs are used to uniquely tag individual DNA molecules, allowing for tracking and correction of errors during sequencing and/or amplification. A UMI may be about 8 base pairs to about 12 base pairs long and may be attached to each DNA molecule before amplification (e.g., PCR). The UMI sequences are random, so each DNA molecule receives a unique tag to distinguish identical DNA sequences from different molecules.

In some embodiments, each primer of the plurality of primers can include an adapter for sequencing using RCA, which amplifies circular DNA (e.g., circularized singled stranded DNA). RCA can generate long repeating sequences called concatemers, which can be used to create accurate consensus sequences, especially for low-input DNA samples or in single-molecule sequencing applications. In some embodiments, extracted DNA sequences, from one or more samples, may be fragmented (e.g., mechanical or enzymatic) and the ends of the DNA fragments either blunted or phosphorylated. Optionally, a linker sequence may be ligated to the DNA ends to facilitate circularization of the DNA fragments. The DNA fragments may be circularized (e.g., using a T4 DNA ligase or similar enzyme). For amplification, one or more primers may bind to a specific region (e.g., taxonomic region) on the circular DNA molecule. The DNA may be amplified, for example using a strand-displacing DNA polymerase (e.g., Phi29 polymerase) that synthesizes new DNA by extending from the primer along the circular template. The DNA polymerase continuously moves around the circular template to produce a long, single-stranded DNA that includes multiple repeats (concatenates) of the original circularized DNA molecule. In some embodiments, RCA occurs under isothermal conditions (e.g., constant temperature), which can result in the generation of long strands with many repeated units (concatenated) of the original circular DNA. By reading the same DNA sequence multiple times in a concatemer (where the same template is repeated), errors introduced during sequencing or polymerase mistakes can be identified and corrected by comparing the repeated sequences (e.g., as in Rolling Circle Amplification to Concatemeric Consensus). For demultiplexing, each repeat is treated as a separate read.

In some embodiments, method 400 may further include determining a biodiversity, phylogenetic diversity, and/or functional diversity of the environmental sample (i.e., biodiversity metrics of a sample). Determining the biodiversity may include determining a taxonomic assignment of the environmental sample. Alternatively, or additionally, determining the biodiversity may include determining a number of species present in the environmental sample. For example, in any sample, there may be zero species to five species, zero species to ten species, ten species to 100 species, 100 species to 1,000 species, 500 species to 1,000,000 species, etc. Determining the biodiversity may include comparing the DNA sequencing data to a database (e.g., database 280) that includes one or more reference sequences or a plurality of reference sequences. A portion of the DNA sequencing data or a subset of the DNA sequencing data may be aligned to one or more or a plurality of reference sequences to determine whether there is overlap in the sequences, homology between the sequences, and the like. When there is overlap or homology, a probable matching identity (or one or more probable matching identities) of the lifeform (e.g., a probable species assignment, a probable genus assignment, a probable family assignment, a probable order assignment, a probable class assignment, a probable phylum assignment, a probable kingdom assignment, a probable domain assignment, a probable taxonomic assignment, etc.) may be output from the system, computing device 270, or other computing device. The probable matching identity may be output with a probability score, for example, to indicate a likelihood that the lifeform is accurate based on the comparison to the reference sequences. In some embodiments, the system may output one or more secondary or backup probable matching identities.

Biodiversity may refer to the species richness or the total number of species in an environment. Phylogenetic diversity may refer to the evolutionary relationships between those species. Two ecosystems with the same number of species (same biodiversity) could have different levels of phylogenetic diversity if one contains species that are more evolutionarily distinct from one another. Functional diversity may refer to the different functional traits present within a community, reflecting the variety of roles that species play in an ecosystem. Two communities with the same species richness could have different levels of functional diversity if the species in one community have a wider range of functional traits. As such, determining a biodiversity of a sample may include determining a phylogenetic diversity of the sample. Determining a biodiversity of a sample may include determining a functional diversity of the sample.

FIG. 6 shows a flow diagram of an embodiment of a computer-implemented method 600 for demultiplexing sequencing data to determine a biodiversity of one or more environmental samples. The method 600 may be executed by a computing device (e.g., computing device 270 of FIG. 2 or optionally processor 252 or processor 263) and includes a series of steps (S610-S690), one or more of which may be optional, that process sequencing data generated from amplicons produced using non-obligate 1:1 primer pair-based pools.

The method 600 may include, at block S610, receiving one or more inputs (e.g., in a configuration file) including sequencing data (e.g., FASTQ files) generated from a sequencing platform (e.g., Illumina®, Oxford Nanopore®) and one or more of: sequences for primers in one or more primer pools, a primer map, and/or a sample guide or map. The sample guide maps sample identifiers (e.g., name, type, alphanumeric code, unique identifier, etc.) to one or more primer pools and associated tag sequences. A sample may be associated with a set of primers or primer pools and at least one tag sequence. A primer pool includes a plurality of primers, including one or more forward primers and one or more reverse primers, that are not constrained to obligate 1:1 primer pairings. The one or more inputs may also define an orientation of each primer, the pool to which a primer belongs, and/or associated sample metadata.

At optional block S620, the method 600 may include performing validation and parsing operations on the one or more inputs. This may include parsing primer pool definitions, validating the sample guide for formatting and content consistency, and/or checking sequence data for integrity and compatibility with downstream processes. The goal of block S620 may be to ensure the one or more inputs meet predefined criteria before proceeding to subsequent stages of the workflow. For example, the predefined criteria may include any one or more of the following, but in no way exclusive, inclusive, or exhaustive:

Input detection and/or normalization may include one or more of:
detect or auto-detect a type of file and/or a type of file encoding (e.g., FASTA, FASTQ, plain versus zip; UTF-8 vs. Windows-1252; etc.);
normalization of one or more headers and/or identity documents (e.g., trim whitespace, collapse multiple spaces, collapse underscores, enforce allowed characters, etc.);
resolve configuration precedence (i.e., deciding which setting to apply when a program or application finds multiple, conflicting configuration values); and/or transmit a final resolved configuration.

Schema and/or cross-file consistency may include one or more of:
sample guide schema including required columns present, unique sample ID, legal characters, no empty cells, etc.;
cross-reference that every pool in the sample guide exists in the primers file; every barcode/index reference exists; no orphan primers or primer pools, etc.; and
uniqueness including no duplicate index pairs per run; detect collisions across primer pools if reuse isn't allowed.

Primer and/or index sanity may include one or more of:
validate primer sequences (e.g., IUPAC codes allowed, mixed case, length bounds, etc.);
compute Hamming/Levenshtein distances within each index set to ensure enough separation for the chosen mismatch tolerance; warn if too close;
check for reverse-complement collisions and/or accidental adapter contamination (e.g., partial adapter in primer string); and
enforce per-pool primer compatibility rules (e.g., pool A uses primer set X only).

Sequence-level preflight checks may include one or more of:
sample a small fraction of reads to estimate length distribution, nucleotide content, and/or over-represented k-mers (i.e., adapter/primer contamination); and
confirm FASTQ quality encoding and presence of quality lines; reject truncated/4-line-cycle breaks, etc.

Parameter sanity checks may include one or more of:
scoring and mismatch thresholds within safe ranges; disallow contradictory flags (e.g., no primer with primer-constrained pools), etc.;
subsample settings coherent with lane/run size; thread/worker counts within system limits; and
fail-fast rules versus lenient/warn-only mode.

Reproducibility and provenance may include one or more of:
record software and dependency versions, reference database/primer set versions, repository hash, if available (take the content supplied to it and return a unique key that could be used to store it in a database); and
capture run metadata (e.g., timestamp, machine, command line, etc.) and hashes of input files for later traceability.

Resource and environment checks may include verifying writable output locations;
sufficient free disk (e.g., estimate from input size and expected split fan-out), etc.

Safety and contamination guards may include one or more of:
block obvious path traversal in sample IDs, sanitize names for file system use, etc.;
flag duplicated tags across different sample IDs; detect swapped pool labels or mixed primer sets, etc.; and
optional negative control or blank sample policy (exists or warn).

Outputs may include one or more of:
normalized, in-memory structures: compiled primer/index automata, pool map, and/or a finalized configuration object; and
a cached file so downstream steps can run without re-validating unless inputs changed.

At optional block S630, the method 600 may include triggering a series of pre-processing operations to prepare the input data for downstream analysis. These operations may include heuristic orientation to determine the likely directionality or structure of the input, validation to ensure the data meets expected formats or criteria (e.g., size, file type, spacing, etc.), and sequence prefiltering to remove or flag sequenced amplicons that do not meet quality thresholds or relevance criteria. These checks help optimize the efficiency and accuracy of subsequent processing steps.

Heuristic orientation may include, but not be limited to, any one or more of the following, not intending to be exhaustive or limiting:

lightweight orientation and seeding may include one or more of:
a fast scan for expected adapter/primer k-mers at read ends (e.g., both strands) to predict forward/reverse orientation;
tag reads with orientation (forward, reverse, unknown), pass as a hint to downstream alignment; and
optional early end-trim of obvious adapter stubs (e.g., without touching biological sequence).

Quick quality and/or length gates may include one or more of:
drop or tag reads with:
missing or short quality lines, non-ACGTN symbols beyond allowed IUPAC allowed codes;
length outside run expectations (e.g., less than about 200 bp or great than about 5 kb for runs);
homopolymers or poly-G tail artifacts (e.g., common on some sequencing platforms); and
maintain counters; do not hard-fail unless thresholds exceeded.

Chimera or concatemer heuristics may include one or more of:
detect internal adapter/primer motifs suggesting concatemer or chimera; and
either tag and route to later split or recovery, or soft-fail with reason that a chimera is suspected.

Inputs to outputs may include one or more of:
input normalized configuration and open FASTQ stream(s); and
output (to index search) reads with light trims and annotations, etc.

Quality thresholds or relevance criteria may include, but not be limited to, any one or more of the following, not intending to be exhaustive:

Length and/or integrity checks may include checking reads for plausible length boundaries before attempting dual-index or primer matching. If they're too short to contain both expected primer regions, they're excluded early. The length and/or integrity checks may be configurable using command line interface arguments or inferred from primer positions. Length and/or integrity checks may be logged in trace tables, for example when debugging is enabled.

Adapter and/or tag contamination and orientation may include inspecting read ends for adapter motifs to determine orientation (forward, reverse, unknown). This may be part of a pre-orientation step and may prevent double-assignment or mis-assignment to a primer pool or primer pair. Adapter and/or tag contamination and orientation may not necessarily trim adapters beyond their role in demultiplexing, but it may use their presence or absence as signals.

Primer search and/or mismatch tolerance may include approximate matching of both tags and primers within predefined mismatch tolerances. Reads missing primer hits or with excessive mismatches may be dropped or marked as “unresolved.” This functions as a quality gate against malformed amplicons.

Cross-pool validation and/or expected index pairing may include excluding reads that match index or primer combinations inconsistent with the declared primer pool. This may ensure biological relevance (i.e., avoids cross-pool contamination). In some embodiments, these events may be logged in “downgrade” or “unresolved” categories.

Conflict resolution heuristics may include handling ambiguous or multiple matched reads (e.g., both forward and reverse indexes match multiple samples) using one or more of: tie-breaking heuristics, “downgrade-full” policy, or demoting to “unassigned.”

Trace logging and diagnostics may include outputting filtering decision(s) (e.g., length, missing primer, cross-pool mismatch, multi-match, etc.), for example when debug modes are used.

At block S640, the method 600 may include demultiplexing the sequencing data based on the tag sequences. Demultiplexing may include assigning each read to a sample-level bin based on the forward and reverse tag sequence combinations. For example, one or more reads originating from a sample may be compared to a library or database of tag sequences so that two or more primers or primer pools may be identified as the amplifying primer pair or primer pool.

At block S650, the method 600 may include performing a secondary demultiplexing of the reads within one or more sample-level bins. The secondary demultiplexing determines valid permutations of forward and reverse primers defined in the primer pool for a given sample. Determining a valid permutation of forward and reverse primers may include identifying optimal primer pairings from a predefined pool of candidate sequences. The matching process may consider valid permutations within the constraints of the pool, such as compatibility rules, thermodynamic properties, and target specificity. Once candidate matches are identified, the system may confirm the directionality of each primer, ensuring that forward and reverse primers are correctly oriented relative to the target sequence. This may ensure that sequences generated and derived from valid, directionally appropriate primer pairs proceed to downstream analysis. A read is assigned to a primer-pair-specific bin based on a best matching primer pair.

For example, once a read is associated with a tag (forward or reverse), the expected primer sequences within that read may be determined. This may include confirming the orientation (forward, reverse, or ambiguous) of a read, and/or trimming primers so that the output sequence begins and ends at biologically meaningful boundaries. This step provides the biological anchor for every demultiplexed read, ensuring that it actually represents the amplicon region intended by the primer set and not random noise or a misassigned index pair. In some embodiments, assigning a read to a primer-pair-specific bin may include a primer lookup based on a primer pool context. For example, each read is already associated with a primer pool (e.g., from the sample guide) during the index search step. The pool defines which forward and reverse primers are valid for that subset of reads. The search space is restricted to those valid primers (e.g., may be about 2 to about 8 total), dramatically speeding up alignment. In some embodiments, identifying primer sequences in a read may be accomplished through approximate matching using, for example, a fast C library for edit-distance alignment. Matching may be approximate, not exact, allowing a few mismatches to accommodate sequencing error, especially at read ends. Both the forward primer and the reverse complement of the reverse primer are searched for. In some embodiments, identifying primer sequences in a read may include primer position validation. Primer position validation ensures that the relative orientation and distance between forward and reverse primers are biologically plausible. For example, a forward primer should occur near the start, and a reverse primer should occur near the end (e.g., within an expected amplicon length). A reverse primer should appear after the forward primer when accounting for strand direction. If reversed or inverted, the orientation of the read is flipped and marked reverse. In some embodiments, identifying primer sequences in a read may include orientation determination. A final orientation of a primer may be decided based on which primer is found first and in what strand. For example, for a forward orientation, a read contains a forward primer in sense direction. For a reverse orientation, a read contains a reverse primer (or complement) in sense direction, so the sequence must be reverse complemented. When both primers are determined to be on the same strand or no clear orientation is determined, these reads may be marked as “unresolved.” This avoids generating false per-sample FASTQs with mixed orientation reads, which impacts directional amplicons. In some embodiments, identifying primer sequences in a read may include primer trimming. Once both primers are found, the read may be trimmed to start immediately after the forward primer and end immediately before the reverse primer. Trimming boundaries may be recorded for trace output. The resulting trimmed amplicon may be used in downstream processing for consensus building or clustering. In some embodiments, identifying primer sequences in a read may include reads that fail primer validation. Reads that fail primer validation may be flagged and retained for optional rescue or excluded if both primers are missing or mismatched beyond a predefined threshold. Common failure reasons include, but are not limited to, missing expected primer motif (e.g., poor basecalling), primer is internal to the read (e.g., chimeric concatemer), and/or multiple conflicting hits (e.g., multi-primer artifact). In some embodiments, identifying primer sequences in a read may include outputting each successfully processed read. A successfully processed read may have a confirmed orientation flag (forward, reverse, unknown), a trimmed sequence coordinate(s), primer match scores and/or mismatches, and/or optional trace entry logging (e.g., primer names, edit distances, hit positions, direction, and pool identification).

At block S660, the method 600 may include determining one or more attributes of the sample based on one or more of: the amplicons that were derived from the primer pool (i.e., which species, class, genus, etc. are present), the tag sequence(s) that are associated with the sample, which primers are associated with the sample or the tag sequence(s), and/or the context of the sample (e.g., metadata associated with the sample, environment from which the sample was taken, etc.). Determining one or more attributes of the sample may include integrating validated contextual signals (e.g., index matches, primer context, orientation, pool metadata, etc.) into a final sample identification or ID (i.e., a sample-level demultiplexing decision)._These attributes may be both a decision logic and a validation checkpoint ensuring reads represent a legitimate, expected combination of identifiers. For example, in some embodiments, each read may include associated data indicating forward or reverse index match(es), primer set(s), orientation status, and/or pool ID (e.g., from the sample guide). Impossible or cross-pool combinations may be filtered out. Samples whose forward and reverse indexes belong to the same pool may be further considered, processed, and/or analyzed. For example, this may ensure that primer set that is used is compatible with the configuration of the particular primer pool. In some embodiments, determining one or more attributes of the sample may include index pairing and match scoring. When multiple potential matches exist (e.g., similar barcodes or off-by-one mismatches), a pairwise match confidence may be calculated. The pairwise match confidence may edit distance for forward and reverse indexes, may perform weighted scoring by combining forward and reverse quality, and/or may include a configurable tie-breaking, for example “retain-best,” “downgrade-full,” or “multi-match discard.” This determines whether the read is uniquely assignable or ambiguous. In some embodiments, determining one or more attributes of the sample may include primer context confirmation. The system may double-check that the primer pair detected earlier corresponds to what the primer pool for the sample expects (e.g., based on the sample guide). This may solve the technical problem of multiple primer pools sharing similar index sets. Reads with primer-pool mismatches are flagged and either downgraded (if secondary valid pairing exists) or excluded (if clearly inconsistent). In some embodiments, determining one or more attributes of the sample may include orientation reconciliation. The detected strand orientation of the read may be cross verified with primer expectations. For example, forward pool primers should yield forward orientation reads, and reverse-paired reads are reverse-complemented before assignment. In some embodiments, determining one or more attributes of the sample may include assignment classification. Each read may be placed into one of several outcome categories. This classification ensures every read either lands in the right sample or contributes to clear diagnostic statistics, not silently misassigned. In some embodiments, determining one or more attributes of the sample may include metadata propagation. When a read is successfully assigned the output filename and/or path may be determined from the sample ID. The record may retain a read ID, index/primer score(s), orientation, run name and/or barcode plate, and/or quality control flags if downgraded. In some embodiments, determining one or more attributes of the sample may include outputting a set of demultiplexed per-sample FASTQ files, a log or summary table describing assignment counts per sample, and/or a trace file capturing read-level decisions and reasons for failure/demotion.

At optional block S670, the method 600 may include generating an output file (e.g., FASTQ file) comprising a per sample set of sequences. The output file may additionally, or alternatively, include one or more partial or unknown bins or sample sequences.

At optional block S680, the method 600 may include providing in the output file or a second output file one or more primer pool statistics and/or visualizations. For example, a read count, a quality metric, etc. may be given for one or more primer pools and/or primer pair combinations. Additionally, or alternatively, the output file or the second output file may include generating a report or visualization summarizing the biodiversity metrics for a sample, including, for example, species richness, taxonomic composition, and/or biodiversity scores or credits.

At block S690, the method 600 may include comparing the demultiplexed reads to a reference database to assign taxonomic identities to the sequences (also called herein amplicons) from one or more samples. Block S690 may include sequence alignment, clustering, and/or amplicon sequence variant (ASV) identification.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor on the thermal cycler 250, DNA sequence 260, computing device 270, and/or server or other computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor (e.g., GPU, CPU, ASIC, FPGA, microcontroller, neural processing unit, DSP, etc.), but any suitable dedicated hardware or hardware/firmware combination can alternatively or additionally execute the instructions.

### Working Example

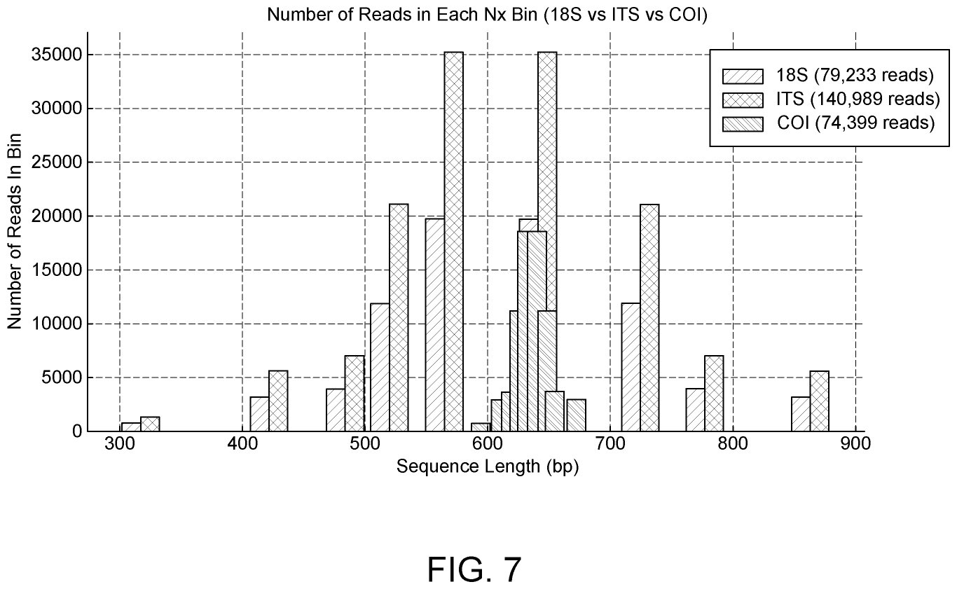

Example 0. FIG. 7 illustrates graphical data showing number of reads per bin per sequence length for primer pools spanning the ITS, COI, and 18S regions (see, e.g., the sequence listing for example primer pairs). Soil was collected from a natural area near Brighton, Michigan, USA. Five soil cores were taken approximately 1 m apart, combined, and homogenized. The composite sample was passed through a standard soil sieve to remove coarse debris and subsequently air-dried using an Excalibur® dehydrator to minimize microbial degradation prior to extraction. Genomic DNA was extracted from the homogenized soil using the IBI Scientific Soil DNA Extraction Kit (IBI Scientific, Dubuque, IA, USA) according to the manufacturer's instructions. Extracted DNA was diluted 1:10 with nuclease-free water prior to use as PCR template.

Amplicon libraries were generated using a primer pool of 21 primers designed to simultaneously target fungal and eukaryotic ribosomal markers. The pool contained multiple primer pools spanning the ITS, COI, and 18S regions (see, e.g., the sequence listing for example primer pairs). Each primer was flanked with unique overhang adapters (e.g., SEQ. ID 57 and SEQ. ID 58). PCR reactions were performed using a master mix supplied by Fortis® Life Sciences (Waltham, MA, USA) and cycling conditions: 95° C. for 3 min; 35 cycles of 95° C. for 30 s, 55° C. for 30 s, 72° C. for 45 s; final extension 72° C. for 5 min. Amplifications were performed in triplicate, pooled, and purified with paramagnetic beads (AMPure-style) prior to quantification.

Purified PCR amplicons were used as input for nanopore library construction with the Oxford Nanopore Technologies Ligation Sequencing Kit LSK114, following manufacturer protocols for short-amplicon preparation. Libraries were sequenced on a Flongle™ flow cell (R10.4.1 chemistry) using a MinION™ Mk1C platform. Sequencing runs were controlled with MinKNOW™ software (v23.04), and raw signal data were basecalled and analyzed using Guppy v7.0.15 in high-accuracy mode.

The graph in FIG. 7 shows that the total read counts between and within each target locus are similar (within an order of magnitude), allowing for an even mix of diverse target organisms to be sequenced and demultiplexed simultaneously. These data exemplify, in at least an embodiment, that the pools of PCR primers are mixed in predefined ratios in order to accomplish specific objectives. As described above, loci may be biased. There are more bacterial DNA than any other organismal group in an environmental DNA sample, as an example. A primer concentration may be lowered to limit the amount of amplification of one species to at least partially unbias the amplification process. Further, for example, within an environmental DNA sample, fungal DNA may be amplified preferentially to plant DNA. Thus, the primer pools or plurality of primers may be in a 20:1 ratio, a 15:1 ratio, a 10:1 ratio, or a 5:1 ratio, of the primers targeting plants versus targeting fungal loci. The data in FIG. 7 show that the amplification was unbiased since the total read counts were similar among the target loci, within an order of magnitude.

Example 1. A kit for determining a biodiversity of an environmental sample, the kit comprising: a multi-well plate, wherein each well of the multi-well plate is configured to receive the environmental sample from a plurality of environmental samples; and a plurality of primer sets complementary to a taxonomic region of a genome, each primer set being disposed in a well of the multi-well plate, wherein: each primer comprises a first sequence configured to be demultiplexed to identify each environmental sample of the plurality of environmental samples of the multi-well plate, and each primer set comprises a plurality of primers comprising one or more forward primers and one or more reverse primers, wherein: the plurality of primers does not comprise obligate 1:1 pairs of forward and reverse primers, the plurality of primers comprises a second sequence that is complementary to a region of a taxonomic group, and is configured to be demultiplexed to identify one or more taxonomic groups of interest in one or more environmental samples of the plurality of environmental samples.

Example 2. The kit of any one of the preceding examples, but particularly Example 1, wherein the plurality of primers is configured to generate amplicons of about 50 base pairs to about 350 base pairs.

Example 3. The kit of any one of the preceding examples, but particularly Example 1, wherein the plurality of primers is configured to generate amplicons of about 500 base pairs to about 5000 base pairs.

Example 4. The kit of any one of the preceding examples, but particularly Example 1, wherein the taxonomic group encompasses plants.

Example 5. The kit of any one of the preceding examples, but particularly Example 1, wherein the taxonomic group encompasses bacteria.

Example 6. The kit of any one of the preceding examples, but particularly Example 1, wherein the taxonomic group encompasses fungus.

Example 7. The kit of any one of the preceding examples, but particularly Example 1, wherein the taxonomic group encompasses fungus and plants.

Example 8. The kit of any one of the preceding examples, but particularly Example 1, wherein the taxonomic group encompasses a first subset of fungal species and a second subset of fungal species.

Example 9. The kit of any one of the preceding examples, but particularly Example 8, wherein there is overlap in species between the first subset of fungal species and the second subset of fungal species.

Example 10. The kit of any one of the preceding examples, but particularly Example 1, wherein the first sequence is a sample tag sequence that comprises an about 8 bp to about 24 bp unique base pair sequence.

Example 11. The kit of any one of the preceding examples, but particularly Example 10, wherein the one or more environmental samples comprises one of: a soil sample, an air sample, a water sample, a gut sample from an organism, a physical specimen of a lifeform, or a sample isolated from a tool having interacted with an element in an environment.

Example 12. The kit of any one of the preceding examples, but particularly Example 1, wherein determining the biodiversity comprises determining a taxonomic assignment of the environmental sample.

Example 13. The kit of any one of the preceding examples, but particularly Example 1, wherein determining the biodiversity comprises determining a number of species present in the environmental sample.

Example 14. The kit of any one of the preceding examples, but particularly Example 1, wherein determining the biodiversity comprises determining a biodiversity metric of the environmental sample.

Example 15. A kit for determining a biodiversity of an environmental sample, the kit comprising: a multi-well plate, wherein each well of the multi-well plate is configured to receive the environmental sample from a plurality of environmental samples; and a plurality of primer sets, each primer set being disposed in a well of the multi-well plate, wherein: each primer set comprises a first sequence comprising a sample tag sequence configured to be demultiplexed to identify each environmental sample of the plurality of environmental samples of the multi-well plate, and each primer set comprises a plurality of primers comprising one or more forward primers and one or more reverse primers, wherein: the plurality of primers does not comprise obligate 1:1 pairs of forward and reverse primers, a first subset of the plurality of primers comprises a first taxonomically informative sequence that is complementary to a first region of a first taxonomic group, a second subset of the plurality of primers comprises a second taxonomically informative sequence that is complementary to a second region of a second taxonomic group, and the first taxonomically informative sequence and the second taxonomically informative sequence are configured to be demultiplexed to identify whether one or both of the first taxonomic group and the second taxonomic group are present in one or more environmental samples of the plurality of environmental samples.

Example 16. The kit of any one of the preceding examples, but particularly Example 15, wherein the plurality of primers is configured to generate amplicons of about 50 base pairs to about 350 base pairs.

Example 17. The kit of any one of the preceding examples, but particularly Example 15, wherein the plurality of primers is configured to generate amplicons of about 500 base pairs to about 5000 base pairs.

Example 18. The kit of any one of the preceding examples, but particularly Example 15, wherein the first taxonomic group encompasses plants, and the second taxonomic group encompasses bacteria.

Example 19. The kit of any one of the preceding examples, but particularly Example 15, wherein the first taxonomic group encompasses plants, and the second taxonomic group encompasses bacteria.

Example 20. The kit of any one of the preceding examples, but particularly Example 15, wherein the first taxonomic group encompasses fungus, and the second taxonomic group encompasses bacteria.

Example 21. The kit of any one of the preceding examples, but particularly Example 15, wherein the first taxonomic group encompasses fungus, and the second taxonomic group encompasses plants.

Example 22. The kit of any one of the preceding examples, but particularly Example 15, wherein the first taxonomic group encompasses a first subset of fungal species, and the second taxonomic group encompasses a second subset of fungal species.

Example 23. The kit of any one of the preceding examples, but particularly Example 22, wherein there is overlap in species between the first subset of fungal species and the second subset of fungal species.

Example 24. The kit of any one of the preceding examples, but particularly Example 15, wherein the sample tag sequence comprises an about 8 bp to about 15 bp unique base pair sequence.

Example 25. The kit of any one of the preceding examples, but particularly Example 15, wherein the environmental sample comprises one of: a soil sample, an air sample, a water sample, a gut sample from an organism, a physical specimen of a lifeform, or a sample isolated from a tool having interacted with an element in an environment.

Example 26. The kit of any one of the preceding examples, but particularly Example 15, wherein determining the biodiversity comprises determining a taxonomic assignment of the environmental sample.

Example 27. The kit of any one of the preceding examples, but particularly Example 15, wherein determining the biodiversity comprises determining a number of species present in the environmental sample.

Example 28. A computer-implemented method, configured to be performed by one or more hardware processors, for determining a biodiversity of an environmental sample, the computer-implemented method comprising: receiving sequenced DNA data based on amplified DNA from an executed PCR, the amplified DNA having been amplified with a plurality of primer sets, wherein: each primer set comprises a plurality of primers comprising one or more forward primers and one or more reverse primers, the plurality of primers does not comprise obligate 1:1 pairs of forward primers and reverse primers, and each primer set is specific to one or more taxonomic regions of target DNA; and demultiplexing the sequenced DNA data to identify at least one taxonomic group, wherein the demultiplexing is based on: a sample tag sequence integrated into each primer set, the sample tag sequence being configured to identify each environmental sample, and each permutation of each primer combination that is present in the plurality of primers specific to an individual barcode region.

Example 29. The computer-implemented method of any one of the preceding examples, but particularly Example 28, further comprising: comparing the sequenced DNA data to a reference database of reference sequences; and outputting an indication of a taxonomic composition of at least one environmental sample based on the demultiplexing the sequenced DNA data and the comparing.

Example 30. The computer-implemented method of any one of the preceding examples, but particularly Example 29, further comprising calculating a biodiversity score or credit based on the indication.

Example 31. The computer-implemented method of any one of the preceding examples, but particularly Example 30, wherein the biodiversity score or credit comprises a taxonomic assignment of the environmental sample.

Example 32. The computer-implemented method of any one of the preceding examples, but particularly Example 28, wherein each primer of the plurality of primers comprises a unique molecular identifier (UMI) for sequencing, and wherein demultiplexing comprises identifying the at least one taxonomic group using each unique molecular identifier in the sequenced DNA data.

Example 33. The computer-implemented method of any one of the preceding examples, but particularly Example 30, wherein each primer of the plurality of primers comprises an adapter for sequencing using Rolling Circle Amplification to Concatemeric Consensus

Example 34. The computer-implemented method of any one of the preceding examples, but particularly Example 30, wherein the taxonomic composition comprises a first taxonomic group based on a first taxonomically informative sequence and a second taxonomic group based on a second taxonomically informative sequence.

Example 35. The computer-implemented method of any one of the preceding examples, but particularly Example 34, wherein the first taxonomically informative sequence is integrated into a first subset of the plurality of primer sets, and the second taxonomically informative sequence is integrated into a second subset of the plurality of primer sets.

### Working Examples of Primer Combinations

SEQ ID NOs. 31-35 may be used in a PCR reaction or a kit to target fungal IT-L taxonomic sequences.

SEQ ID NOs. 31-35 and SEQ ID NOs. 55-56 may be used in a PCR reaction or a kit to target fungal IT-S taxonomic sequences.

SEQ ID NOs. 1-21 may be used in a PCR reaction or kit to target fungal 16S, 18S, ITS, and 28S taxonomic sequences.

SEQ ID NOs. 22-24 may be used in a PCR reaction or kit to target fungal 16S, 18S, ITS, and 28S taxonomic sequences.

SEQ ID NOs. 25-26 may be used in a PCR reaction or kit to target fungal 16S, 18S, ITS, and 28S taxonomic sequences.

SEQ ID NOs. 27-30 may be used in a PCR reaction or kit to target fungal 16S, 18S, ITS, and 28S taxonomic sequences.

SEQ ID NOs. 31-36 may be used in a PCR reaction or kit to target plant taxonomic sequences.

SEQ ID NOs. 37-40 may be used in a PCR reaction or kit to target plant taxonomic sequences.

SEQ ID NOs. 41-48 may be used in a PCR reaction or kit to target plant taxonomic sequences.

SEQ ID NOs. 49-54 may be used in a PCR reaction or kit to target plant taxonomic sequences.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “sequence” or “primer” may include, and is contemplated to include, a plurality of sequences or a plurality of primers. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A kit for determining a biodiversity of an environmental sample, the kit comprising:
a multi-well plate, wherein each well of the multi-well plate is configured to receive the environmental sample from a plurality of environmental samples; and
a plurality of primer sets complementary to a taxonomic region of a genome, each primer set being disposed in a well of the multi-well plate, wherein:
each primer comprises a first sequence configured to be demultiplexed to identify each environmental sample of the plurality of environmental samples of the multi-well plate, and
each primer set comprises a plurality of primers comprising a plurality of forward primers and a plurality of reverse primers, wherein:
the plurality of primers does not comprise obligate 1:1 pairs of forward and reverse primers,
the plurality of primers comprises a second sequence that is complementary to a region of a taxonomic group, and is configured to be demultiplexed to identify one or more taxonomic groups of interest in one or more environmental samples of the plurality of environmental samples.

2. The kit of claim 1, wherein the plurality of primers is configured to generate amplicons of about 50 base pairs to about 350 base pairs.

3. The kit of claim 1, wherein the plurality of primers is configured to generate amplicons of about 500 base pairs to about 5000 base pairs.

4. The kit of claim 1, wherein the taxonomic group encompasses plants.

5. The kit of claim 1, wherein the taxonomic group encompasses bacteria.

6. The kit of claim 1, wherein the taxonomic group encompasses fungus.

7. The kit of claim 1, wherein the taxonomic group encompasses fungus and plants.

8. The kit of claim 1, wherein the taxonomic group encompasses a first subset of fungal species and a second subset of fungal species.

9. The kit of claim 8, wherein there is overlap in species between the first subset of fungal species and the second subset of fungal species.

10. The kit of claim 1, wherein the first sequence is a sample tag sequence that comprises an about 8 bp to about 24 bp unique base pair sequence.

11. The kit of claim 10, wherein the one or more environmental samples comprises one of: a soil sample, an air sample, a water sample, a gut sample from an organism, a physical specimen of a lifeform, or a sample isolated from a tool having interacted with an element in an environment.

12. The kit of claim 1, wherein determining the biodiversity comprises determining a taxonomic assignment of the environmental sample.

13. The kit of claim 1, wherein determining the biodiversity comprises determining a number of species present in the environmental sample.

14. The kit of claim 1, wherein determining the biodiversity comprises determining a biodiversity metric of the environmental sample.

15. The kit of claim 1, wherein a first subset of the plurality of primers targeting a first taxonomic group of the one or more taxonomic groups is present in a first ratio, and a second subset of the plurality of primers targeting a second taxonomic group of the one or more taxonomic groups is present in a second ratio, the second ratio being different from the first ratio.

16. The kit of claim 15, wherein the first ration and the second ratio are selected from the group consisting of: a 20:1 ratio, a 15:1 ratio, a 10:1 ratio, or a 5:1 ratio loci.

17. A kit for determining a biodiversity of an environmental sample, the kit comprising:
a multi-well plate, wherein each well of the multi-well plate is configured to receive the environmental sample from a plurality of environmental samples; and
a plurality of primer sets, each primer set being disposed in a well of the multi-well plate, wherein:
each primer set comprises a first sequence comprising a sample tag sequence configured to be demultiplexed to identify each environmental sample of the plurality of environmental samples of the multi-well plate, and
each primer set comprises a plurality of primers comprising two or more forward primers and two or more reverse primers, wherein:
the plurality of primers does not comprise obligate 1:1 pairs of forward and reverse primers,
a first subset of the plurality of primers comprises a first taxonomically informative sequence that is complementary to a first region of a first taxonomic group,
a second subset of the plurality of primers comprises a second taxonomically informative sequence that is complementary to a second region of a second taxonomic group,
the first subset of the plurality of primers is present in a first ratio, and the second subset of the plurality of primers is present in a second ratio, the second ratio being different from the first ratio, and
the first taxonomically informative sequence and the second taxonomically informative sequence are configured to be demultiplexed to identify whether one or both of the first taxonomic group and the second taxonomic group are present in one or more environmental samples of the plurality of environmental samples.

18. The kit of claim 17, wherein the environmental sample comprises one of: a soil sample, an air sample, a water sample, a gut sample from an organism, a physical specimen of a lifeform, or a sample isolated from a tool having interacted with an element in an environment.

19. The kit of claim 17, wherein determining the biodiversity comprises determining a taxonomic assignment of the environmental sample.

20. The kit of claim 17, wherein determining the biodiversity comprises determining a number of species present in the environmental sample.
